SEQUENCE LISTING (60 sequences)

SEQ ID NO: 1 - DNA, 20 bp, Artificial Sequence
  primer
tgatgtccgaactgaagcag

SEQ ID NO: 2 - DNA, 20 bp, Artificial Sequence
  primer
tcgatatccgtctggaggtc

SEQ ID NO: 3 - DNA, 20 bp, Artificial Sequence
  primer
ccaaaaccaaaacggagaaa

SEQ ID NO: 4 - DNA, 20 bp, Artificial Sequence
  primer
ttcctcctcctccctatcgt

SEQ ID NO: 5 - DNA, 20 bp, Artificial Sequence
  primer
gctggaagacttggaagcac

SEQ ID NO: 6 - DNA, 20 bp, Artificial Sequence
  primer
tcacaatgcctattccacga

SEQ ID NO: 7 - DNA, 20 bp, Artificial Sequence
  primer
attctggctgcaggactgtt

SEQ ID NO: 8 - DNA, 20 bp, Artificial Sequence
  primer
aagcctccatgggagagaat

SEQ ID NO: 9 - DNA, 20 bp, Artificial Sequence
  primer
agacactcgtcaggcaacct

SEQ ID NO: 10 - DNA, 20 bp, Artificial Sequence
  primer
attgacgcgttctctttgct

SEQ ID NO: 11 - DNA, 20 bp, Artificial Sequence
  primer
aacccagaggcattgacaac

SEQ ID NO: 12 - DNA, 20 bp, Artificial Sequence
  primer
attgtggaccaggaacttgc

SEQ ID NO: 13 - DNA, 20 bp, Artificial Sequence
  primer
caaggggaaggaaaagaagg

SEQ ID NO: 14 - DNA, 20 bp, Artificial Sequence
  primer
gaggactcatctcggtcagc

SEQ ID NO: 15 - DNA, 20 bp, Artificial Sequence
  primer
cctgccgaagatgaagagtc

SEQ ID NO: 16 - DNA, 20 bp, Artificial Sequence
  primer
ttcggttccaacaacaaaca

SEQ ID NO: 17 - DNA, 20 bp, Artificial Sequence
  primer
tgttgctgccaaagaaattg

SEQ ID NO: 18 - DNA, 20 bp, Artificial Sequence
  primer
gctagtccagcacacaccaa

SEQ ID NO: 19 - DNA, 20 bp, Artificial Sequence
  primer
cacgaagagccagagagctt

SEQ ID NO: 20 - DNA, 20 bp, Artificial Sequence
  primer
catgtcgggaaccaggtagt

SEQ ID NO: 21 - DNA, 20 bp, Artificial Sequence
  primer
agctaggggaagctcctgtc

SEQ ID NO: 22 - DNA, 20 bp, Artificial Sequence
  primer
ggcggctctgagttgtagac

SEQ ID NO: 23 - DNA, 20 bp, Artificial Sequence
  primer
ttttgcctggacaaagaagc

SEQ ID NO: 24 - DNA, 20 bp, Artificial Sequence
  primer
tccgagctgtctcgtctttt

SEQ ID NO: 25 - DNA, 20 bp, Artificial Sequence
  primer
agccatttcccgcttgtact

SEQ ID NO: 26 - DNA, 20 bp, Artificial Sequence
  primer
cctgagggcagctcaatctc

SEQ ID NO: 27 - DNA, 20 bp, Artificial Sequence
  primer
tacacacgagctgcttccac

SEQ ID NO: 28 - DNA, 20 bp, Artificial Sequence
  primer
ctgggtctgacaccgtttct

SEQ ID NO: 29 - DNA, 20 bp, Artificial Sequence
  primer
gtcgagcctgtctgaaaagg

SEQ ID NO: 30 - DNA, 20 bp, Artificial Sequence
  primer
gtgtcttccagctcctcgtc

SEQ ID NO: 31 - DNA, 20 bp, Artificial Sequence
  primer
gagatgcccaagaatgtcgt

SEQ ID NO: 32 - DNA, 20 bp, Artificial Sequence
  primer
cctgccagagtagcttgtcc

SEQ ID NO: 33 - DNA, 20 bp, Artificial Sequence
  primer
aactgacagcccaaaaccac

SEQ ID NO: 34 - DNA, 20 bp, Artificial Sequence
  primer
actgctgaaggctggtgtct

SEQ ID NO: 35 - DNA, 20 bp, Artificial Sequence
  primer
tctgaagaagcagagcagca

SEQ ID NO: 36 - DNA, 20 bp, Artificial Sequence
  primer
accagttgtttggcctgttc

SEQ ID NO: 37 - DNA, 20 bp, Artificial Sequence
  primer
gaagctgaccttgtggaagc

SEQ ID NO: 38 - DNA, 20 bp, Artificial Sequence
  primer
ccggcaatgtagatctggtt

SEQ ID NO: 39 - DNA, 20 bp, Artificial Sequence
  primer
tggtctggtctacgcagttg

SEQ ID NO: 40 - DNA, 20 bp, Artificial Sequence
  primer
gggtgtcgagcatcttcaat

SEQ ID NO: 41 - DNA, 20 bp, Artificial Sequence
  primer
ggaggctttccagttctgct

SEQ ID NO: 42 - DNA, 20 bp, Artificial Sequence
  primer
ctgcgatcccctctccctat

SEQ ID NO: 43 - DNA, 19 bp, Artificial Sequence
  primer
ccagcaaactatcagttaa

SEQ ID NO: 44 - DNA, 19 bp, Artificial Sequence
  primer
ccagtcaacagcaaactca

SEQ ID NO: 45 - DNA, 19 bp, Artificial Sequence
  primer
tggagacgacgatcagaaa

SEQ ID NO: 46 - DNA, 19 bp, Artificial Sequence
  primer
tcactgccctgcacatcaa

SEQ ID NO: 47 - DNA, 23 bp, Artificial Sequence
  primer
cagccccgagtgaggagagtagc

SEQ ID NO: 48 - DNA, 20 bp, Artificial Sequence
  primer
cccccatcgacccccagttt

SEQ ID NO: 49 - DNA, 20 bp, Artificial Sequence
  primer
caatacttctggacttttca

SEQ ID NO: 50 - DNA, 20 bp, Artificial Sequence
  primer
cttttcctggagactttatg

SEQ ID NO: 51 - DNA, 22 bp, Artificial Sequence
  primer
ttctacttcagcaggccttcaa

SEQ ID NO: 52 - DNA, 22 bp, Artificial Sequence
  primer
atattggaagaacttgcccacg

SEQ ID NO: 53 - DNA, 20 bp, Artificial Sequence
  primer
ctgttgaccgatggactcct

SEQ ID NO: 54 - DNA, 20 bp, Artificial Sequence
  primer
gccacagcgtcatatcatcc

SEQ ID NO: 55 - DNA, 21 bp, Artificial Sequence
  primer
tccttgctgacgaccctacat

SEQ ID NO: 56 - DNA, 21 bp, Artificial Sequence
  primer
tacctcaggggatagccttgg

SEQ ID NO: 57 - DNA, 23 bp, Artificial Sequence
  primer
tcttagtagccttagctctcagg

SEQ ID NO: 58 - DNA, 21 bp, Artificial Sequence
  primer
tgtcagtcccatctacaggca

SEQ ID NO: 59 - DNA, 22 bp, Artificial Sequence
  primer
gctgtaccagtgcaggtcctca

SEQ ID NO: 60 - DNA, 22 bp, Artificial Sequence
  primer
catttccacctttgcccaagtc

(19) United States
(12) Patent Application Publication
(10) Pub. No.: US 2021/0163883 A1
(43) Pub. Date: Jun. 3, 2021

(54) IGF2-CONTAINING MEDIUM FOR CULTURING MAMMALIAN EMBRYOS IN VITRO AND CULTURE METHOD

(71) Applicants: SHANDONG UNIVERSITY, Jinan (CN); SDIVF R&D CENTRE LIMITED, Hong Kong (CN)

(72) Inventors: Zijiang CHEN, Jinan (CN); Hongbin LIU, Jinan (CN); Yueran ZHAO, Jinan (CN); Jinlong MA, Jinan (CN); Ling GENG, Jinan (CN); Gang LU, Jinan (CN); Hui LIU, Jinan (CN); Chuanxin ZHANG, Jinan (CN); Mengjing LI, Jinan (CN)

(21) Appl. No.: 17/172,005

(22) Filed: Feb. 9, 2021

Related U.S. Application Data
(63) Continuation-in-part of application No. PCT/CN2019/099117, filed on Aug. 2, 2019.

(30) Foreign Application Priority Data
Aug. 9, 2018 (CN) 201810907126.5

(51) Int. Cl. C12N 5/073

(52) U.S. Cl. CPC C12N 5/0604; C12N 2501/105

(57) ABSTRACT

An in vitro culture medium that can be utilized for culturing mammalian embryos, especially early-stage embryos, is provided. The culture medium comprises about 10-200 nM insulin-like growth factor 2 (IGF2). A method for culturing mammalian embryos in vitro is also provided, which substantially includes culturing an early stage embryo of a mammal using the culture medium. The in vitro culture medium and method can increase the formation rate of blastocysts of mammals, particularly humans, and can also improve the quality of embryos, thereby improving the success rate of assisted reproductive technologies. The culture medium and method are particularly useful in culturing embryo from an aged mammal or a mammal with obesity.

### CROSS-REFERENCE TO RELATED APPLICATIONS

The present application is a continuation of International Patent Application No. PCT/CN2019/099117 filed Aug. 2, 2019, which claims priority to Chinese Patent Application No. 201810907126.5, filed Aug. 9, 2018. The disclosure of these above applications is hereby incorporated into the present application by reference in their entirety.

### REFERENCE TO SEQUENCE LISTING SUBMITTED ELECTRONICALLY

The content of the electronically submitted sequence listing, with the file name culture_ST25.txt, size 11,241 bytes, and date of creation Feb. 9, 2021, filed herewith, is incorporated herein by reference in its entirety.

### FIELD OF THE INVENTION

The invention relates to the field of assisted reproduction of mammals, especially humans. Specifically, the present invention relates to a method and culture medium for in vitro cultivation of mammalian, especially human embryos.

### BACKGROUND

In the past few decades, in vitro fertilization-embryo transfer (IVF-ET) has provided an important solution to the infertility problem of humans and other species. Embryos need to be cultured between fertilization and embryo transfer. Existing technologies have insufficient developmental capabilities of oocytes and embryos, causing slow development after early embryo transfer and even pregnancy failure. Approximately half of human preimplantation embryos stagnate in vitro before reaching the blastocyst stage, which is the stage used for in vivo embryo transfer. Miscarriage and recurrent pregnancy loss characterized by poor embryonic growth are a common human reproductive disorder. The loss of pregnancy in the early embryonic preimplantation period is considered primary infertility.

Embryonic dysplasia and reduced implantation may be caused by chromosomal abnormalities and suboptimal culture conditions. This provides the impetus for the research on the conditions of in vitro culture of oocytes and embryos in IVF-ET. Understanding the effects of growth factors involved in the development of human embryos is very important to explore the conditions of in vitro culture, which will help improve embryo viability and increase the success rate of IVF-ET.

Mammalian eggs, the most important cells in the female body, remain in a quiescent state, but upon fertilization they are reprogrammed into highly specialized totipotent zygotes. This reprogramming sets the embryo onto a developmental course through highly specialized, proliferative states and increasingly differentiated stages ultimately resulting in the development of a new individual. During the oocyte-to-embryo transition, asymmetrical meiotic divisions are replaced by symmetrical meiotic divisions, and cytoplasmic organelle rearrangement and transcriptome modifications are directed by the maternal-zygotic configuration. The maternal-zygotic transition (MZT) is driven by maternal deposition of RNAs and proteins and is a crucial step in early embryonic development, and this occurs 2-3 days post fertilization in mice. Zygotic genome activation (ZGA) is considered a key event during the MZT in early embryonic development. The onset of ZGA varies in species, occurring at the 2-cell-stage in mice and the 4-8-cell-stage in humans. Failure or inappropriate initiation of ZGA leads to developmental arrest, usually at the 2-cell-stage.

One of the key events during embryogenesis is the transition from oocytes to zygotes, which occurs through transcriptional and epigenetic regulation that relies on maternal proteins. A cluster of imprinted genes, including IGF2 and reciprocally imprinted H19 at chromosome 11p15.5, have been shown to play critical roles in fetal and postnatal growth in humans. IMP2 is an imprinted gene that encodes a member of the conserved family of mRNA-binding proteins (IMPs). The IMP2 protein contains two RNA-recognizing motifs that regulate RNA splicing, transport, and IGF2 translation. Transcriptome analyses have shown high levels of expression of the IMP2 protein in oocytes and early pre-implanted embryos in humans and mice, and IMP2 is directly involved in tissue differentiation and fetal growth progression and is associated with energy metabolism, adhesion, the movement of smooth muscles, and the development of type 2 diabetes. Moreover, recent studies have demonstrated a pivotal role for IMP2 in enhancing mRNA stability and translation. However, the role of maternal IMP2 in early embryonic development in mice remains elusive.

Therefore, the art also needs a method that can enhance the survival rate and developmental potential of embryos in the in vitro cultivation of embryos of mammals, especially humans, and a medium for culturing embryos in vitro.

### SUMMARY OF THE INVENTION

The present invention proves for the first time that insulin-like growth factor 2 (IGF2) is essential for the regulation and activation of genes involved in zygotic genome activation of ZGA, and it is found that supplementing IGF2 in the culture medium improves the early embryonic development of mammals, especially humans. The efficiency of development and blastocyst formation improves embryo viability. Thus, the inventor provides a method for in vitro cultivation of mammals, especially human embryos, and a medium for in vitro cultivation of mammals, especially human embryos.

The present invention provides an embryo culture medium comprising IGF2. The culture medium provided by the present invention can be used for culturing an early stage embryo of a mammal.

Insulin-like growth factor 2, IGF2, is encoded by the gene Igf2, which is one of the earliest discovered endogenous imprinted genes. IGF2 is a multifunctional cell proliferation regulator, which plays an important role in promoting cell differentiation and proliferation.

In the present invention, the mammal can be any mammal, including and not limited to rodents (such as mice and rats), lagomorphs (rabbits), carnivores (felines and canines), artiodactyl Orders (bovines and swines), Perissodactyls (eques), or primates and apes (humans or monkeys). The mammal is preferably a human being, a monkey, a rat or a mouse.

In some embodiments, the embryo culture medium in the present invention is for in vitro cultivation or manipulation of a gamete, embryo or stem cell. For example this may include transfer of gametes during and after collection or transferring embryos for implantation.

In one embodiment, the present invention provides a medium for culturing mammalian embryos in vitro, which contains about 10-200 nM IGF2. In another embodiment, the embryo culture medium contains about 25-100 nM IGF2. In another embodiment, the embryo culture medium contains about 45-55 nM IGF2. In yet another embodiment, the embryo culture medium contains about 50 nM IGF2. In another embodiment, the content of IGF2 refers to the working concentration, that is, the concentration in an embryo/cell culture environment. In some cases, the IGF2 in the medium of the present invention is present in a multiple of the working concentration. For example, in order to facilitate storage or operation, the culture medium is provided at 5 times or 10 times the working concentration of the substance contained in it, and water/solution/culture solution is added for dilution during use.

The term “embryo” as used herein may have a broad definition, which includes the pre-embryo phase. The term “embryo” as used herein may encompass all developmental stages from the fertilization of the oocyte through compaction, morula, blastocyst stages, hatching and implantation. In some cases the term “embryo” is used to describe a fertilized oocyte after implantation in the uterus until 8 weeks after fertilization at which stage it become a fetus in humans. According to this definition the fertilized oocyte is often called a pre-embryo until implantation occurs. However as noted above the term “embryo” as used herein may include the pre-embryo phase.

During embryonic development, blastomere numbers increase geometrically (1-2-4-8-16-etc.). Synchronous cell cleavage is generally maintained to the 8-cell stage in human embryos. After that, cell cleavage becomes asynchronous and finally individual cells possess their own cell cycle. Human embryos produced during infertility treatment are usually transferred to the recipient before 8-blastomere stage. In some cases human embryos are also cultivated to the blastocyst stage before transfer. This is preferably done when many good quality embryos are available or prolonged incubation is necessary to await the result of a pre-implantation genetic diagnosis (PGD). However, there is a tendency towards prolonged incubation as the incubation technology improves.

Accordingly, the term embryo is used in the following to denote each of the stages fertilized oocyte, zygote, 2-cell, 4-cell, 8-cell, 16-cell, compaction, morula, blastocyst, expanded blastocyst and hatched blastocyst, as well as all stages in between (e.g. 3-cell or 5-cell).

In one embodiment, the medium according to the present invention is for cultivating an early stage mammal embryo, which is selected from the group of fertilized oocyte, zygote, 2-cell, 4-cell, 8-cell, 16-cell, compaction, morula and blastocyst. In yet another embodiment, the medium according to the present invention is for cultivating a mammal embryo of 2-cell stage. Cultivation medium for embryo in different stage may comprise different nutrition or growth factors which is suitable for the embryo in a specific stage.

Suitably the medium according to the present invention may further comprise one or more additional compounds, e.g. an inorganic salt, an energy source, an amino acid, a protein source, a cytokine, a chelating agent, an antibiotic, a hyaluronan, a growth factor, a hormone, a vitamin and/or a granulocyte-macrophage colony-stimulating factor (GM-CSF).

In one embodiment the culture medium may comprise an inorganic salt. In one embodiment the inorganic salt may be one which dissociates into their inorganic ions in aqueous solution. Suitably the inorganic salt may be one which comprises one or more of the following inorganic ions: Na⁺, K⁺, Cl⁻, Ca²⁺, Mg₂, SO₄²⁻, PO₄³⁻.

The energy source may be pyruvate, lactate or glucose depending on the developmental stage of the embryo. Energy source requirements evolve from a pyruvate-lactate preference while the embryos, up to the 8-cell stage, are under maternal genetic control, to a glucose based metabolism after activation of the embryonic genome that supports their development from 8-cells to blastocysts.

The protein source may be albumin or synthetic serum (e.g. at a concentration of 5 to 20% w/v or v/v respectively). Suitable sources for protein supplementation include human serum, human cord serum (HCS), human serum albumin (HSA), fetal calf serum (FCS) or bovine serum albumin (BSA).

In one embodiment the one or more additional compounds may include a buffer solution. Suitable buffer solutions include HEPES buffer or MOPS buffer for example.

The one or more additional compounds may be in the form of a medium designed to support an embryo (e.g. a mammalian embryo) to grow, which medium could be referred to as a background medium. The background medium may be any medium suitable for the culture of an embryo, a gamete or a stem cell, such as those commercially available basic medium or supplemental medium. In one embodiment the background medium may be one or more of the group consisting of gamete handling medium (including gamete collection medium), a medium for intracytoplasmic sperm injection (ICSI), a fertilization medium, single step embryo culture medium, embryo transfer medium, oocyte maturation medium, sperm preparation and fertilisation medium, or any other suitable medium used for gametes or embryos. Examples of background medium include G-1™, G-2™, HSA-Solution™, G-MOPS™ Plus, G-MOPS™, Embryo Glue™, ICSI™ or G-TL™ or a combination thereof, which can be obtained from Vitrolife AB, Sweden.

In one embodiment, the background medium is M2 Medium (Sigma-Aldrich, Inc. #M7167) or M16 Medium (Sigma-Aldrich, Inc. # M7292). Preferably, the background medium is a M16 Medium, which has components as listed below:
Components g/L
Calcium Chloride.2H₂O 0.25137
Magnesium Sulfate (anhydrous) 0.1649
Potassium Chloride 0.35635
Potassium Phosphate, Monobasic 0.162
Sodium Bicarbonate 2.101
Sodium Chloride 5.53193
Albumin, Bovine Fraction V 4.0
D-Glucose 1.0
Phenol Red.Na 0.0106
Pyruvic Acid.Na 0.0363
DL-Lactic Acid.Na 2.95

In one embodiment, the embryo culture medium of the present invention does not comprise plasminogen and urokinase plasminogen activator.

In one embodiment, the embryo culture medium of the present invention is for the use of cultivating an embryo from an aged mammal. In the present invention, an aged mammal generally refers to a mammal in a late reproductive age or child-bearing age, or even pass the reproductive age. For example, for a human being, a woman of reproductive age is usually between the ages of 12- and 51-years old, while an age older than 30 years, or older than 32 years, or older than 38 years, is deemed a late reproductive age. As another example, for a mouse in a late reproductive age is generally a mouse older than 10 months.

In one embodiment, the embryo culture medium of the present invention is for the use of cultivating an embryo from a mammal suffering of obesity. Obesity a condition that is characterized by excessive accumulation and storage of fat in the body. Weight that is higher than what is considered as a healthy weight for a given height is described as overweight or obese. As an example of standards, body mass index, or BMI, is used as a screening tool for overweight or obesity. Obesity is frequently subdivided into categories based on BMI:
Class 1: BMI of 30 to <35
Class 2: BMI of 35 to <40
Class 3: BMI of 40 or higher. Class 3 obesity is sometimes categorized as “extreme” or “severe” obesity.

The present invention also provides a method for culturing a mammalian embryo in vitro, in which the method includes adding IGF2 in a medium culturing an early stage embryo of a mammal in vitro. In one embodiment, about 10-200 nM IGF2 is added in the medium. In another embodiment, about 25-100 nM IGF2 is added in the medium. In another embodiment, about 45-55 nM IGF2 is added in the medium. In yet another embodiment, about 50 nM IGF2 is added in the medium.

In one embodiment, the method according to the present invention is for cultivating an early stage mammal embryo, which is selected from the group of fertilized oocyte, zygote, 2-cell, 4-cell, 8-cell, 16-cell, compaction, morula and blastocyst. In yet another embodiment, the method according to the present invention is for cultivating a mammal embryo of 2-cell stage. Cultivation medium for embryo in different stage may comprise different nutrition or growth factors which is suitable for the embryo in a specific stage.

In one embodiment, in the method according to the present invention, the culture medium does not comprise plasminogen and urokinase plasminogen activator.

In one embodiment, the method according to the present invention is for cultivating a mammal embryo from an aged mammal. In the present invention, an aged mammal generally refers to a mammal in a late reproductive age or child-bearing age, or even pass the reproductive age.

In one embodiment, the method according to the present invention is for cultivating a mammal embryo from a mammal suffering of obesity.

The present invention also provides a use of IGF2 in preparing a composition for culturing a mammalian embryo in vitro. In one embodiment, said composition contains about 10-200 nM IGF2. In another embodiment, said composition contains about 25-100 nM IGF2. In another embodiment, said composition contains about 45-55 nM IGF2. In yet another embodiment, said composition contains about 50 nM IGF2. In one embodiment, the composition according to the present invention is for cultivating an early stage mammal embryo, which is selected from the group of fertilized oocyte, zygote, 2-cell, 4-cell, 8-cell, 16-cell, compaction, morula and blastocyst. In yet another embodiment, the composition according to the present invention is for cultivating a mammal embryo of 2-cell stage.

### BRIEF DESCRIPTION OF THE DRAWINGS

FIGS. 1A-1C show expression of IMP2 in mouse oocytes and early embryos, with FIG. 1A showing qRT-PCR results for mRNA levels of Imp2 in mouse oocytes and early embryos. Error bars indicate the SEM; FIG. 1B showing immunofluorescent staining of IMP2 in mouse oocytes and preimplantation embryos (scale bar, 10 μm); and FIG. 1C showing Western blot for IMP2 expression in oocytes and early embryos. GCs, granulosa cells. ERK1/2 is used as the protein loading control.

FIGS. 2A-2D show characterization of Imp2 mutant mice, wherein FIG. 2A shows that Imp2 transcripts were detected in control but not Imp2^{-/-} ovaries by semi-quantitative RT-PCR using β-actin as the control for the integrity of the RNA samples (Exon 3 was deleted in the Imp2 knockout strategy); FIG. 2B shows that IMP2 protein was detected in control, but not Imp2^{-/-} MII lysates by immunoblot using antibodies against IMP2 and ACTB (as loading control, and lysate of 100 oocytes in each lane); FIG. 2C shows ovarian histology of control and Imp2^{-/-} females with hematoxylin and eosin stain (CL, corpus luteum; scale bar, 100 μm); and FIG. 2D shows morphology of MII oocytes from control and Imp2^{-/-} females after superovulation at postnatal day 23 (females (n=10) were used for each genotype. Scale bar, 100 μm).

FIGS. 3A-3D show maternal deletion of IMP2 causes impaired early embryogenesis, wherein FIG. 3A shows that maternal IMP2 deletion inhibits early embryonic development. n>10 mice for each genotype; FIG. 3B shows that maternal IMP2 deletion causes impaired blastocyst formation (numbers of embryos (n) flushed in vivo are indicated; n>5 mice for each genotype); FIG. 3C shows morphology of Imp2 female embryos cultured in vitro after mating with WT males (embryonic development was monitored over the indicated time frame after hCG administration; scale bar, 100 μm); and FIG. 3D shows cumulative numbers of pups per female during the defined time period. n>7 mice for each genotype.

FIGS. 4A-4E show that maternal Imp2-knockout zygotes are defective in the MZT, with FIG. 4A showing a schematic diagram for the late 2-cell-stage, control embryos (Imp2^{♀+/♂+}) and Imp2-knockout embryos (Imp2^{♀-/♂+}) for RNA sequencing (20 embryos per group, 3 replicates) and HPLC MS/MS (330 embryos per group, 3 replicates), with PAR, photoactivatable ribonucleoside-enhanced crosslinking and immunoprecipitation; FIG. 4B showing a volcano plot for the downregulated and upregulated genes in 2-cell-stage Imp2-knockout embryos in fold change (x-axis) and statistical significance (−log 10 of the p-value, y-axis), with different dots indicate upregulated and downregulated proteins and RNAs in the merged RNA-protein data; FIG. 4C showing Western blot of 2-cell-stage embryos from control and Imp2^{-/-} female mice probed with antibodies against CCAR1, DDX21, ILF1, FBL, RPS14, IMP2, and ACTB; FIG. 4D showing gene ontology analysis of the downregulated genes in Imp2^{♀-/♂+} embryos compared with WT embryos at the 2-cell-stage; and FIG. 4E showing quantitative real-time PCR (qRT-PCR) analysis showing the expression of transcripts in control and Imp2^{♀-/♂+} embryos at the 2-cell-stage, with error bars indicating the SEM.

FIGS. 5A-5G show that Ccar1 and Rps14 are key target genes of IMP2 that mediate early embryonic developmental potential, with FIG. 5A showing luciferase reporter activity of the indicated downregulated gene-promoters (RLA, relative luciferase activity; Error bars indicate the SEM); FIG. 5B showing luciferase reporter activity of hCCAR1 promoters in response to IGF2BP2; FIG. 5C showing luciferase reporter activity of mCcar 1 promoters in response to Igf2bp2 (Error bars indicate the SEM); FIG. 5D showing a schematic diagram showing the microinjection of early mouse zygotes and subsequent embryo analysis at the molecular and developmental level; FIG. 5E showing that blastocyst development is defective after injecting siRNAs targeting the Ccar1 and Rps14 genes at the indicated times compared with control siRNA (scale bar, 100 μm); FIG. 5F showing quantification of morula (56 h) and blastocyst (80 h) formation after injecting control siRNA or siRNAs targeting Ccar1 and Rps14 (the numbers of embryos (n) analyzed are indicated; error bars indicate the SEM; **p<0.01, Student's t-test); and FIG. 5G showing qRT-PCR results for the expression of IMP2 target genes in 2-cell-stage embryos after Ccar1/Rps14 deletion in zygotes (error bars indicate the SEM).

FIGS. 6A-6D show deletion of IMP2 limits the transcriptional and translational activity in early cleaved embryos, with FIG. 6A showing confocal images of newly synthesized RNA by EU staining in control and 2-cell-stage Imp2^{-/-} female embryos (scale bar, 20 μm); FIG. 6B showing quantification of newly synthesized RNA in control and Imp2^{-/-} female 2-cell-stage embryos by EU incorporation. More than ten embryos were observed for each genotype with six replicates (n=6 mice for each genotype); FIG. 6C showing confocal image indicating the protein synthesis in control and Imp2^{-/-} female 2-cell-stage embryos incorporating HPG (scale bar, 20 μm); and FIG. 6D showing quantification of nascent protein synthesis by HPG incorporation in control and Imp2^{-/-} female 2-cell-stage embryos, and more than ten embryos were observed for each genotype with six replicates (n=5 mice for each genotype).

FIGS. 7A-7E show that IMP2 activates the IGF2 signaling pathway and increases embryonic developmental competency, with FIG. 7A showing schematic diagram for IGF2 treatment of early embryos in M16 medium in vitro; FIG. 7B showing that IGF2 treatment triggers the expression of IMP2 target genes in 2-cell-stage embryos (error bars indicate the SEM); FIGS. 7C and 7D respectively showing morphology (FIG. 7C) and quantification (FIG. 7D), with results showing that IGF2 treatment increases the early embryonic developmental efficiency of control embryos but has no effect on Imp2^{♀-/♂+} embryos (the numbers of embryos (n) analyzed are indicated; n>15 mice for both genotypes; error bars indicate the SEM; *p<0.05 and **p<0.001, Student's t-test; NS, not significant; NT, no treatment; scale bar, 100 μm); and FIG. 7E showing the embryo transfer experiments demonstrating greater rates of embryos development to term after IGF2 treatment (the number of pups per mother is on the left and percentage of pregnant mice is on the right; n represent the pregnant females on the left and total number of foster mothers used on the right; error bars indicate the SEM. *p<0.05, Student's t-test).

FIGS. 8A-8C show that IGF2 improves in vitro embryonic development in humans, with FIG. 8A showing the time line of human oocyte maturation to early embryo growth up to the blastocyst stage, highlighting critical times between the stages and the predicted in vitro culture development in medium with and without IGF2 after intracytoplasmic sperm injection (red arrows indicate the time duration of IGF2 treatment from zygote to blastocyst formation); FIG. 8B showing improved blastocyst formation of human embryos after IGF2 treatment (total numbers of zygotes (n) used are indicated); and FIG. 8C showing morphology of embryos after in vitro culture with or without IGF2 in the culture medium (scale bar, 100 μm).

FIGS. 9A-9B show generation of Imp2^{-/-} mice and oocyte, with FIG. 9A showing schematic diagram of the gene-targeting vector for creating the conditional Imp2-knockout mouse (loxp recombination sites (red triangles) along with a flanked neomycin-selection cassette were introduced flanking exon 3); and FIG. 9B showing that MII oocytes were recovered from hormonally stimulated control (n=10) and Imp2^{-/-} (n=10) females at 16 h after hCG administration (NS, no statistical difference in Student's t-test, p>0.05).

FIGS. 10A-10F show that Imp2 is dispensable for fertilization and early cleavage, with FIG. 10A showing immunofluorescence results for control and Imp2^{-/-} MII oocytes (dashed circles represent the outlines of the oocytes; scale bar, 10 μm.); FIG. 10B showing that after hormonal stimulation and in vivo mating with wild-type males, 1-cell and 2-cell-stage embryos were flushed from control and Imp2^{-/-} female oviducts at embryonic days 0.5 and 1.5. n>5 mice for each genotype (scale bar, 100 μm); FIG. 10C showing that deletion of maternal Imp2 causes impaired morula and blastocyst formation in Imp2^{-/-} females in vitro (n>7 mice for each genotype; error bars show the SEM; **p<0.01, Student's t-test); FIG. 10D showing morphology of embryos collected from the uteri of control and Imp2^{-/-} female mice at embryonic days 2.5 and 3.5 after successful mating with adult WT males (scale bar, 1000 μm; n>6 for both genotypes); FIG. 10E for quantification, which shows that the 50 nM IGF2 concentration is optimum for early embryonic development (n>10 mice were used; error bars indicate the SEM); and FIG. 10F showing photographs for the healthy pups delivered by the IGF2 treatment group after embryo transfer.

FIGS. 11A-11G show luciferase reporter activity of downregulated genes, with FIGS. 11A-11E showing luciferase reporter activity of indicated gene promoters for RPS14 (FIG. 11A), ILF2 (FIG. 11B), DDX21 (FIG. 11C), FBL (FIG. 11D), and HNRNPM (FIG. 11E) in response to IGF2BP2 in human (error bars indicate the SEM); FIGS. 11F and 11G showing luciferase reporter activity of Fbl (FIG. 11F) and Hnrnpm (FIG. 11G) promoters in response to Igf2bp2 in mice (translation was estimated by an increase in luciferase activity after incubation at 30° C. for 30 min; error bars indicate the SEM).

FIGS. 12A and 12B show reduced serum IGF2 protein levels and reduced Igf2 expressions in oocytes from aged mice, with FIG. 12A showing serum IGF2 concentration in young and aged mice assessed via ELISA (n=3 for each group); and FIG. 12B showing qPCR results for mRNA levels of Igf2 and target genes in GV-stage and MII-stage oocytes from young and aged mice (Student's t-test (two-tailed). *p<0.05; error bars indicate the SEM).

FIGS. 13A-13F show IGF2 administration in culture medium improves the oocytes maturation and early embryonic developmental competence of aged mice, with FIG. 13A showing schematic diagram of IGF2-treatment of oocytes and early embryos in M16 medium in vitro; FIGS. 13B and 13C showing quantitative analysis of GVBD (FIG. 13B) and Pb1 extrusion in control oocytes (n=164) and IGF2-treated oocytes (n=180) (FIG. 13C); FIG. 13D showing quantitative analysis of blastocysts in control embryos (n=218) and IGF2-treated embryos (n=222); FIG. 13E showing morphology of in vitro cultured oocytes and embryos examined for development within specific time frames (arrows indicate the oocytes which failed to extrude a polar body; arrowheads denote embryos which failed to develop into blastocysts; scale bar, 100 μm); and FIG. 13F showing quantitative analysis of the pregnancy rate in the control and IGF2-treated embryos (15 blastocysts were transferred into the uterus of each female; n here indicates the numbers of females used as recipients; *p<0.05, Student's t-test (two-tailed); NS, not significant).

FIGS. 14A-14E show that IGF2 ameliorates the meiotic defects of aged mouse oocytes, with FIG. 14A showing representative images of spindle/chromosome organization in control and IGF2-treated oocytes from aged mice (spindles were stained with an antibody against α-tubulin (green), and chromosomes were counter-stained with Hoechst 33342 (blue); scale bar=30 μm); FIG. 14B showing quantification of abnormal spindle/chromosomes oocytes in control (n=95) and IGF2-treated (n=105) oocytes groups (a Student's t-test (two-tailed), *p<0.05; error bars indicate the SEM); FIG. 14C showing representative images of CM-H2DCFDA fluorescence (green) in control and IGF2-treated oocytes (scale bar=20 μm); FIG. 14D showing quantification of ROS signals in control oocytes (n=25) and IGF2-treated oocytes (n=21) (a Student's t-test (two-tailed), *p<0.05; error bars indicate the SEM); and FIG. 14E showing adenosine triphosphate (ATP) contents in control oocytes (n=50) and IGF2-treated oocytes (n=50) (a Student's t-test (two-tailed), *p<0.05; error bars indicate the SEM).

FIGS. 15A-15F show that IGF2 improves the mitochondrial functional activity of oocytes from aged mice, with FIG. 15A showing that mitochondria were stained with mitotracker Green FM (green) (scale bar=20 μm); FIG. 15B showing quantification of mitochondrial distribution signals in control oocytes (n=26) and IGF2-treated oocytes (n=25) (a Student's t-test (two-tailed), *p<0.05; error bars indicate the SEM); FIG. 15C showing JC-1 staining for the mitochondrial membrane potential (MMP) in control and IGF2-treated oocytes; FIG. 15D showing quantification of the red/green fluorescence intensity ratio in control oocytes (n=40) and IGF2-treated oocytes (n=35) (a Student's t-test (two-tailed), *p<0.05; error bars indicate the SEM); FIG. 15E showing HPG Fluorescent staining for total protein synthesis in MII-stage oocytes with or without IGF2-treatment (oocytes were incubated in M16 medium with 50 μM HPG for 1 h prior to staining; scale bar=30 μm); and FIG. 15F showing quantification of HPG signal intensity in control (n=28) and IGF2-treated (n=29) oocytes (*p<0.05, a Student's t-test (two-tailed); error bars indicate the SEM).

FIGS. 16A-16C show that IGF2 improves the mitochondrial ultrastructure of oocytes from aged mice, with FIG. 16A showing representative TEM micrographs of mitochondria from control and IGF2-treated oocytes at 2,500× magnification (scale bar=1 μm; note the normal (Mn) and vacuolated (Mv) mitochondria); FIG. 16B showing quantification of mitochondria per defined region of interest (ROI) in control and IGF2-treated oocytes. n=9 oocytes for each group (a Student's t-test (two-tailed), *p<0.05; error bars indicate the SEM); FIG. 16C showing representative TEM micrographs of mitochondria from control and IGF2-treated oocytes at 60,000× magnification (inner membrane (IM), outer membrane (OM), and intermembrane space (IMS); scale bar=200 nm).

FIGS. 17A-17E show that IGF2 reduces the apoptosis and promotes the level of autophagy in aged mouse oocytes, with FIG. 17A showing LC3 staining, which shows the extent of autophagy occurring in control and IGF2-treated oocytes; FIG. 17B showing quantification of LC3 intensity in control (n=34) and IGF2-treated oocytes (n=25) (a Student's t-test (two-tailed), *p<0.05; error bars indicate the SEM); FIG. 17C showing TUNEL assay of control and IGF2-treated oocytes from aged mice (a green fluorescence signal indicates TUNEL-positive oocytes; Apoptotic signals were observed after 16 h of in vitro culture; DNA was counterstained with DAPI; scale bar=30 μm); FIG. 17D showing the percentage of apoptosis-positive oocytes in control (n=61) and IGF2-treated oocytes group (n=44) (a Student's t-test (two-tailed), *p<0.05; error bars indicate the SEM); and FIG. 17E showing qPCR results for mRNA levels of Sirt1, Bmp15, Gdf9, and Sod1 in MII-stage oocytes after in vitro maturation with or without IGF2-treatment (*p<0.05, A Student's t-test (two-tailed); error bars indicate the SEM).

FIGS. 18A-18B show obese mice have reduced serum IGF2 protein levels and their oocytes have reduced Igf2 expression, with FIG. 18A showing the IGF2 level in blood sera samples from ND and HFD mice using ELISA (the HFD mice had significantly reduced IGF2 concentrations); and FIG. 18B showing a qPCR analysis of GV-stage and MII-stage oocytes retrieved from ND and HFD mice, which revealed reductions in the mRNA levels of Igf2, Bmp15, Sod1, Gdf9, and Gpx4.

### DETAILED DESCRIPTION

The technical details and benefits of the invention provided in the present disclosure are further described in the following examples, which are intended to illustrate the inventions and not to limit the scope of the present disclosure.

### Example 1 Ethics Approval

This study was approved by the Institutional Review Board (IRB) of Reproductive Medicine of Shandong University. Experiments related to humans were in accordance with the ethical standards of the institutional research committee. Before participation, all the candidates provide the written, informed consent.

### Example 2 Materials and Methods

Oocyte/Embryo Collection and Microinjection

Mice that were 24-28 days old were superstimulated with 5 IU pregnant mare's serum gonadotropin (PMSG) followed by 5 IU human chorionic gonadotrophin (hCG) for 44 h. Oocytes were collected and cultured in small drops of M16 medium (M7292; Sigma-Aldrich) and were covered with mineral oil and maintained in 5% CO₂ at 37° C. For collection of zygotes and embryos, control and Imp2^{-/-} females were mated with adult WT males post hCG injection. For the collection of zygotes oviducts were punctured while for embryos collection uteri were flushed at the indicated time points after hCG administration. For microinjection, mRNAs were transcribed in vitro with the mMESSAGE mMACHINE SP6 Transcription kit (Invitrogen, AM1450). siRNA was obtained from RiboBio, and the sequences are mentioned in Table 2.

Zygote Culture, Embryo Transfer, and Fertility Assessment Test

Zygotes were cultured in small-drop of KSOM medium (Sigma-Aldrich) at 37° C. in 5% CO₂ for observing their embryonic developmental potential. For the microinjection-related experiment, the embryos were cultured in G-1 and G-2 media (Sigma-Aldrich).

For the IGF2 protocol, for zygotes culture M16 medium with or without 50 nM IGF2 (CF61, Novoprotein) was used. Embryonic development and morphology were examined with a stereomicroscope (Nikon SMZ1500).

Blastocysts obtained with and without IGF2 treatment were used for embryo transfer. A total 19 pseudopregnant Kunming female mice were used as the recipients (16 embryos were transferred to the uterus of each mouse). The pregnancy rates to term and the litter sizes were recorded.

For in vivo validation of fertility, control and Imp2^{-/-} females were caged with adult WT males for a period of 6 months. Fertility was assessed by the number of pups per female during the defined time period. More than ten females were allocated for each genotype, and more than five cages were set for the experiment.

Culture of Human Zygotes with IGF2 Treatment

Spare human GV oocytes of good morphology were collected and matured in vitro in 5% CO₂, 5% O₂, and 90% N₂ at 37° C. After maturation, MII oocytes were used for the ICSI protocol. Zygotes with intact morphology were allocated to the control and experimental groups. Zygotes were cultured with or without 50 nM IGF2 (CF61, Novoprotein) and incubated in 5% CO₂, 5% O₂, and 90% N₂ at 37° C. The assessment of embryonic development and embryo quality was recorded, and photomicrographs were taken at the blastocyst stage.

For RNA-sequencing, late 2-cell-stage embryos were collected from control and Imp2^{-/-} females (20 embryos per group, 3 replicates). RNA-sequencing protocol was carried out. Briefly, total RNA was isolated from embryo samples using the RNAeasy mini kit (Qiagen) according to the manufacturer's protocols. mRNA-RFP was added to calculate the mRNA copy number. NEB Next Ultra RNA library prep kit for Illumina was applied for generating sequencing library by using extracted total RNA. Library was sequenced by Hiseq 2000 and aligned RNA-sequence reads to Mus musculus UCSC mm9 references with the Tophat software (http://tophat.cbcb.umd.edu/).

HPLC MS/MS analysis was carried out Briefly, embryos at late 2-cell-stage were collected from control and Imp2^{-/-} females (330 embryos per group, 3 replicates). Protein extraction buffer was used for the lysis of embryos that contain 75 mM NaCl, 50 mM Tris (pH 8.2), 8 M urea, 1 mM NaF, 1% (v/v) EDTA-free protease inhibitor cocktail, 1 mM βglycerophosphate, 1 mM sodium orthovanadate, 10 mM sodium pyrophosphate, and 1 mM PMSF. Lysates were centrifuged at 40,000 g for 1 h, and Bradford assay was used to measure the protein contents. To reduce the cysteine residues, 5 mM DTT was used at 56° C. for the duration of 25 min followed by alkylated in 14 mM iodoacetamide at room temperature for 30 min. Samples were digested overnight with trypsin using enzyme-to-substrate ratio 1:200 and then peptide were divided into aliquots. After that samples were subjected for TMT labelling. Aliquots of the same samples were combined, lyophilized and resuspended in buffer A (10 mM ammonium acetate, pH 10) having volume 110 μl, and then loaded onto a XBridge™ BEH130 C18 column (2.1×150 mm, 3.5 μm; Waters) with the UltiMate® 3000 HPLC systems at a flow rate of 200 μl/min. For MS evaluation, 30 fractions were sequentially resuspended in 0.1% FA and LTQ Orbitrap Velos mass spectrometer (Thermo Finnigan, San Jose, Calif.) coupled on-line to a Proxeon Easy-nLC 1000 was used for analysis. Peptides were loaded onto a trap column (75 μm×2 cm, Acclaim® PepMap100 C18 column, 3 μm, 100 Å; DIONEX, Sunnyvale, Calif.) at a flow rate of 10 μl/min, and transferred to a reverse-phase microcapillary column (75 μm×25 cm, Acclaim® PepMap RSLC C18 column, 2 μm, 100 Å; DIONEX, Sunnyvale, Calif.) at a flow rate of 300 nl/min. The HPLC solvent A and solvent B was used. A 205-min linear gradient was used for protein identification and quantification. Gene ontology analysis of gene enrichment was measured using the Database for Annotation, Visualization and Integrated Discovery.

Confocal Microscopy

Oocytes and early embryos were fixed in 4% PBS mixed with paraformaldehyde for 30 min. Oocytes/embryos were blocked in 1% BSA dissolved in PBS and incubated with primary antibodies diluted in blocking solution for 1 h and followed by incubation with secondary antibodies for 30 min after several washes and then counter-stained with 5 μg/ml DAPI (4′,6-diamidino-2-phenylindole, Life Technologies) for 10 min. After mounting, oocytes/embryos were examined with a confocal laser scanning microscope (Zeiss LSM 780, Carl Zeiss AG, Germany). The antibodies used in these experiments are shown in Table 3

Histological Analysis

Paraffin-embedded ovary samples were fixed in 10% formalin overnight at 4° C., deparaffinized, sectioned at a thickness of 5 μm, and stained with hematoxylin and eosin. Images were obtained under an optical microscope.

Cell Culture, Plasmid Transfection, and Luciferase Assay

For the growth of HEK293 cells, DMEM/high glucose (Hyclone) containing 10% fetal bovine serum was used, and cells were incubated at 37° C. with 5% CO₂. Transient plasmid transfections were performed using the X-treme-GENE HP DNA Transfection Reagent (Roche). For the luciferase assay, luciferase reporters were used with or without plasmids encoding components of IGF2BP2 for cell transfection. Secreted alkaline phosphatase expression was used as the loading control. The supernatant from cultured HEK293 cells was collected after 48 h and used for the luciferase assay according to the manufacturer's instructions (Dual Luciferase System, GeneCopoeia).

EU Incorporation Assays

EU corporation assays was performed by using Click-iT RNA Imaging kits (C10329, Invitrogen). Two-cell-stage embryos from both genotypes (control and Imp2^{-/-}) were collected. Embryos were incubated in culture medium supplemented with 1 mM 5′ EU (ethynyl uridine) for 3 h prior to Hoechst 33342 staining according to the kit's instructions. Laser scanning confocal microscope was used for Images detection.

Detection of Protein Synthesis

Control and Imp2-deleted 2-cell-stage embryos were incubated in culture medium supplemented with in 50 μM HPG (L-homopropargylglycine) for 2 h. Embryos were incubated at 37° C. with 5% CO₂ for 30 min and then washed with PBS. Formaldehyde (3.7%) was used for fixation followed by permiabilization with 0.5% Triton X-100 for 30 min at room temperature. HPG was detected using the Click-iT protein synthesis assay kit (C10428, Life Technolgies).

RNA Extraction and qRT-PCR Validation

RNeasy mini kit (Qiagen) was used for the extraction of total RNA following the manufacturer's protocol. Genomic DNA was removed by digesting with the RNase-free genomic DNA eraser buffer (Qiagen), and cDNA was obtained by reverse transcription of RNA using PrimeScript™ reverse transcriptase (Takara). Power SYBR Green Master Mix (Takara) was used on a Roche 480 PCR system for qRT-PCR analysis. The mRNA level was calculated by normalizing to the endogenous mRNA level of actin (internal control) using Microsoft Excel. The qRT-PCR reactions were performed in triplicate for each experiment using gene-specific primers. Primer sequences are shown in Table 2.

Western Blot Analysis

For total protein extraction, 100 oocytes or embryos were lysed and separated by SDS PAGE and transferred to a PVDF membrane (Millipore). The membrane was incubated with primary antibody followed by HRP-conjugated secondary antibody, and bands were examined using an Enhanced Chemiluminescence Detection Kit (Bio-Rad). The antibodies used are shown in Table 3.

Statistical Analysis

Results are shown as the means±SEM, and at least three replicates were included for each experiment. Comparisons were made by two-tailed unpaired Student's t-tests, and p-values<0.05 were considered significant.

TABLE 1
Protein and genes

| | Human/ | |
|---|---|---|
| Gene Symbol | mouse | Gene name |
| CCAR1 | Human | cell division cycle and apoptosis regulator 1 |
| Ccar1 | mouse | cell division cycle and apoptosis regulator 1 |
| FBL | human | Fibrillarin |
| Fbl | mouse | Fibrillarin |
| HNRNPM | human | Heterogeneous nuclear ribonucleoprotein M |
| Hnrnpm | mouse | heterogeneous nuclear ribonucleoprotein M |
| FYTTD1 | human | forty-two-three domain containing 1 |
| ILF2 | human | interleukin enhancer binding factor 2 |
| RPS14 | human | ribosomal protein S14 |
| DDX21 | human | DEXD-box helicase 21 |
| RPL32 | human | Ribosomal protein L32 |
| PHGDH | human | phosphoglycerate dehydrogenase |
| PSAT1 | human | phosphoserine aminotransferase 1 |
| HNRNPA2B1 | human | heterogeneous nuclear ribonucleoprotein A2/B1 |

TABLE 2
Primers and usages thereof

| Gene | | |
|---|---|---|
| name | Primers Sequence | Usage |
| Ddx21 | F: 5′-TGATGTCCGAACTGAAGCAG-3′ (SEQ ID NO: 1) | Real-time |
| | R: 5′-TCGATATCCGTCTGGAGGTC-3′ (SEQ ID NO: 2) | |
| Ccar1 | F: 5′-CCAAAACCAAAACGGAGAAA-3′ (SEQ ID NO: 3) | |
| | R: 5′-TTCCTCCTCCTCCCTATCGT-3′ (SEQ ID NO: 4) | |
| Hnrnpm | F: 5′-GCTGGAAGACTTGGAAGCAC-3′ (SEQ ID NO: 5) | |
| | R: 5′-TCACAATGCCTATTCCACGA-3′ (SEQ ID NO: 6) | |
| Ilf2 | F: 5′-ATTCTGGCTGCAGGACTGTT-3′ (SEQ ID NO: 7) | |
| | R: 5′-AAGCCTCCATGGGAGAGAAT-3′ (SEQ ID NO: 8) | |
| Fyttd1 | F: 5′-AGACACTCGTCAGGCAACCT-3′ (SEQ ID NO: 9) | |
| | R: 5′-ATTGACGCGTTCTCTTTGCT-3′ (SEQ ID NO: 10) | |
| Rpl32 | F: 5′-AACCCAGAGGCATTGACAAC-3′ (SEQ ID NO: 11) | |
| | R: 5′-ATTGTGGACCAGGAACTTGC-3′ (SEQ ID NO: 12) | |
| Rps14 | F: 5′-CAAGGGGAAGGAAAAGAAGG-3′ (SEQ ID NO: 13) | |
| | R: 5′-GAGGACTCATCTCGGTCAGC-3′ (SEQ ID NO: 14) | |
| Gtf2i | F: 5′-CCTGCCGAAGATGAAGAGTC-3′ (SEQ ID NO: 15) | |
| | R: 5′-TTCGGTTCCAACAACAAACA-3′ (SEQ ID NO: 16) | |
| Mpc2 | F: 5′-TGTTGCTGCCAAAGAAATTG-3′ (SEQ ID NO: 17) | |
| | R: 5′-GCTAGTCCAGCACACACCAA-3′ (SEQ ID NO: 18) | |
| Set | F: 5′-CACGAAGAGCCAGAGAGCTT-3′ (SEQ ID NO: 19) | |
| | R: 5′-CATGTCGGGAACCAGGTAGT-3′ (SEQ ID NO: 20) | |
| Sf1 | F: 5′-AGCTAGGGGAAGCTCCTGTC-3′ (SEQ ID NO: 21) | |
| | R: 5′-GGCGGCTCTGAGTTGTAGAC-3′ (SEQ ID NO: 22) | |
| Pgrmc1 | F: 5′-TTTTGCCTGGACAAAGAAGC-3′ (SEQ ID NO: 23) | |
| | R: 5′-TCCGAGCTGTCTCGTCTTTT-3′ (SEQ ID NO: 24) | |
| Nat10 | F: 5′-AGCCATTTCCCGCTTGTACT-3′ (SEQ ID NO: 25) | |
| | R: 5′-CCTGAGGGCAGCTCAATCTC-3′ (SEQ ID NO: 26) | |
| Rps19 | F: 5′-TACACACGAGCTGCTTCCAC-3′ (SEQ ID NO: 27) | |
| | R: 5′-CTGGGTCTGACACCGTTTCT-3′ (SEQ ID NO: 28) | |
| Usp10 | F: 5′-GTCGAGCCTGTCTGAAAAGG-3′ (SEQ ID NO: 29) | |
| | R: 5′-GTGTCTTCCAGCTCCTCGTC-3′ (SEQ ID NO: 30) | |
| Mrp14 | F: 5′-GAGATGCCCAAGAATGTCGT-3′ (SEQ ID NO: 31) | |
| | R: 5′-CCTGCCAGAGTAGCTTGTCC-3′ (SEQ ID NO: 32) | |
| Dnttip2 | F: 5′-AACTGACAGCCCAAAACCAC-3′ (SEQ ID NO: 33) | |
| | R: 5′-ACTGCTGAAGGCTGGTGTCT-3′ (SEQ ID NO: 34) | |
| Nelfe | F: 5′-TCTGAAGAAGCAGAGCAGCA-3′ (SEQ ID NO: 35) | |
| | R: 5′-ACCAGTTGTTTGGCCTGTTC-3′ (SEQ ID NO: 36) | |
| Hnrnp1 | F: 5′-GAAGCTGACCTTGTGGAAGC-3′ (SEQ ID NO: 37) | |
| | R: 5′-CCGGCAATGTAGATCTGGTT-3′ (SEQ ID NO: 38) | |
| Fbl | F: 5′-TGGTCTGGTCTACGCAGTTG-3′ (SEQ ID NO: 39) | |
| | R: 5′-GGGTGTCGAGCATCTTCAAT-3′ (SEQ ID NO: 40) | |
| Phgdh | F: 5′-GGAGGCTTTCCAGTTCTGCT-3′ (SEQ ID NO: 41) | |
| | R: 5′-CTGCGATCCCCTCTCCCTAT-3′ (SEQ ID NO: 42) | |
| Ccar1 | F: 5′ CCAGCAAACTATCAGTTAA-3′ (SEQ ID NO: 43) | siRNA |
| | R: 5′ CCAGTCAACAGCAAACTCA-3′ (SEQ ID NO: 44) | |
| Rps14 | F: 5′ TGGAGACGACGATCAGAAA-3′ (SEQ ID NO: 45) | |
| | R: 5′ TCACTGCCCTGCACATCAA-3′ (SEQ ID NO: 46) | |
| Imp2 + | F: 5′-CAGCCCCGAGTGAGGAGAGTAGC-3′ (SEQ ID NO: | Geno- |
| flox- | 47) | typing |
| 62 | R: 5′-CCCCCATCGACCCCCAGTTT-3′ (SEQ ID NO: 48) | |
| Imp2 | F: 5′-CAATACTTCTGGACTTTTCA-3′ (SEQ ID NO: 49) | |
| Δ-50 | R: 5′-CTTTTCCTGGAGACTTTATG-3′ (SEQ ID NO: 50) | |

TABLE 3
antibodies

| Protein | Manufacture | Applications |
|---|---|---|
| name | (catalogue number) | (working dilution) |
| IMP2 | Cell signaling (14672) | WB(1:1000) |
| IMP2 | Abcam (ab124930) | IF(1:50) |
| CCAR1 | Gentex (GTX110892) | WB(1:200) |
| FBL | Abcam (ab166630) | WB(1:250) |
| RPS14 | Proteintech (16683-1-AP) | WB(1:100) |
| DDX21 | Santa cruz (sc-376785) | WB(1:50) |
| ILF2 | Abcam (ab154169) | WB(1:300) |
| ACTIN | Cell signaling (4970) | WB(1:1000) |

### Example 3 High Expression of Imp2 in Mouse Oocytes and Early Embryos

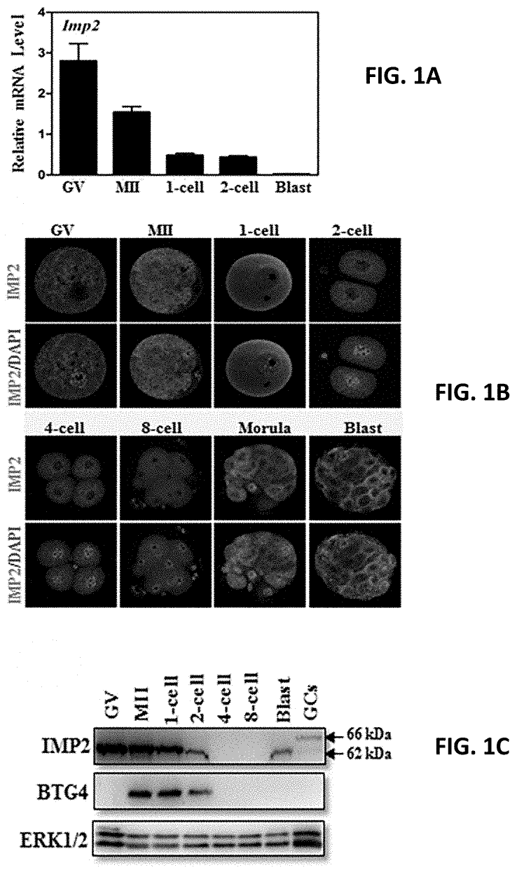

Protein and mRNA profiles of mouse IMP2 in oocytes and early embryos were determined by western blot and quantitative real-time PCR (qRT-PCR), respectively. We found that transcripts of the mRNA-binding protein IMP2 were highly expressed in mouse oocytes and early-stage embryos. Expression was greatest at the germinal vesicle (GV) stage, and it was significantly decreased in MII oocytes. Expression was further reduced after fertilization and was completely absent by the blastocyst stage (FIG. 1A).

Immunofluorescence staining showed that IMP2 was localized in the cytoplasm of oocytes and pre-implantation embryos (FIG. 1B). IMP2 expression was evenly distributed in oocyte stages but underwent dynamic changes during zygote development. The morula and blastocyst stages showed the expression of IMP2 at the outer edges of blastomeres (FIG. 1B), and western blot analysis further confirmed the presence of the IMP2 protein in oocytes and early embryos (FIG. 1C). Collectively, these findings indicate that IMP2 is highly expressed during the MZT.

### Example 4 Characterization of Imp2-Knockout Mice

To investigate the physiological function of IMP2, a conditional Imp2-knockout mouse was generated by flanking exons 3 and 4 of the Imp2 allele with LoxP sites (FIG. 9A).

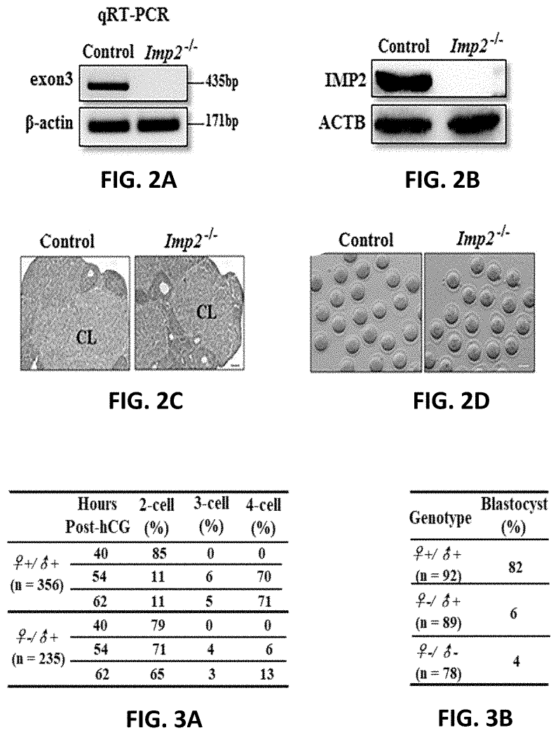

Imp2 transcript expression was abolished in Imp2^{-/-} ovaries and egg lysates (FIGS. 2A and 2B). Imp2^{-/-} females had normal folliculogenesis and corpora lutea and were indistinguishable from control mice (FIG. 2C).

To further examine the role of IMP2 in oogenesis, MII oocytes were recovered after gonadotropin administration. The numbers and morphologies of MII oocytes derived from Imp2^{-/-} female mice showed no significant difference compared with controls (FIG. 2D and FIG. 9B). These findings suggest that Imp2 is not required for oocyte maturation or ovulation.

### Example 5 Deletion of Maternal Imp2 Results in Early Embryonic Developmental Arrest

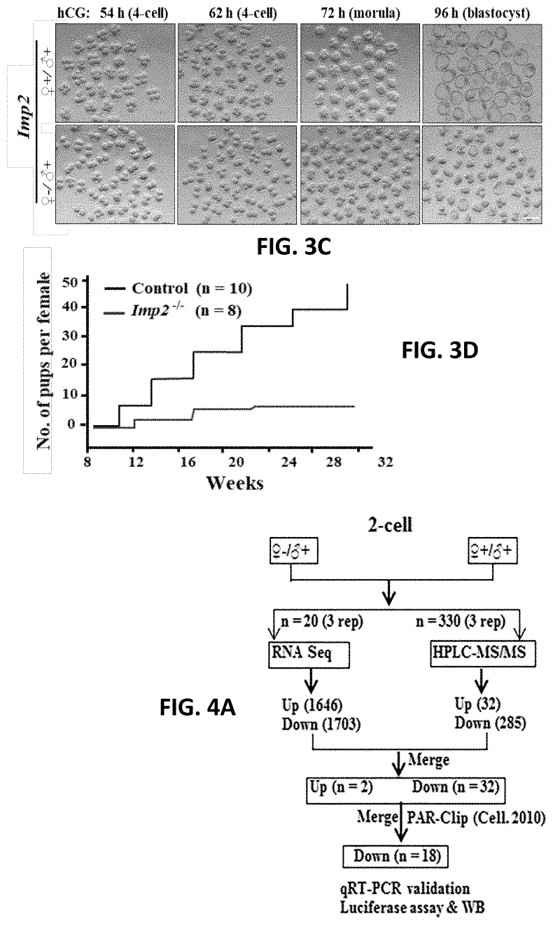

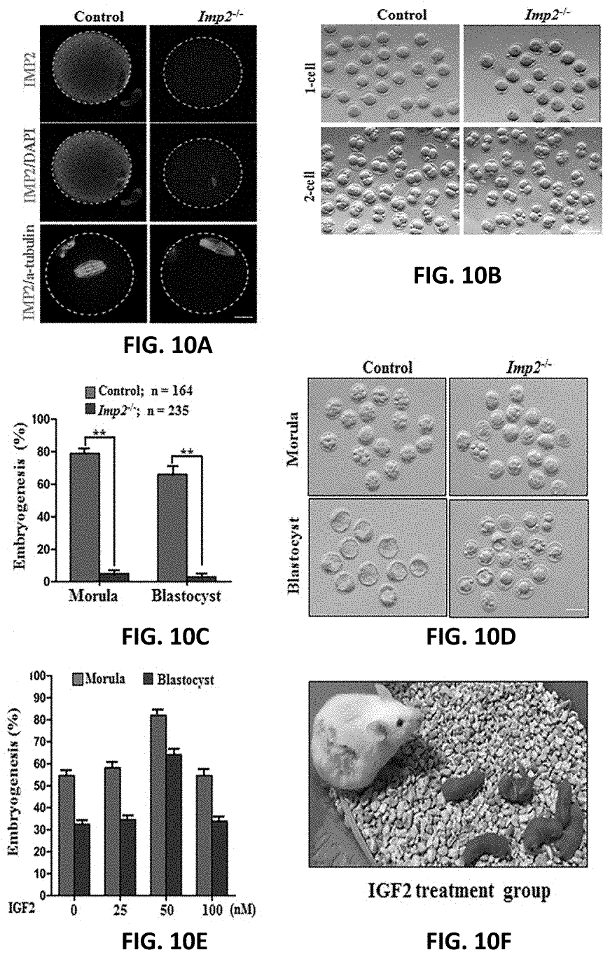

To investigate the role of IMP2 in early embryonic development, Imp2 was deleted in female germline cells at different stages of oocyte development. IMP2 expression was abolished at the oocyte stage as a result of the knockout of the Imp2 gene (FIG. 10A). To understand the contribution of Imp2 to embryonic development, control and Imp2^{-/-} females were mated with wild-type males. Control female zygotes (Imp2^{♀+/♂+}) and Imp2^{-/-} female zygotes (Imp2^{♀-/♂+}) were obtained and then cultured in vitro after successful matting. No significant difference was observed in the development or morphology of zygotes or 2-cell-stage embryos (FIG. 3A and FIG. 10B). However, Imp2^{-/-} female embryos (Imp2^{♀-/♂+}) had prolonged 2-cell-stages with 71% of the embryos arrested at the 2-cell-stage at 54 hours post-human chorionic gonadotrophin (hCG) compared to 11% of control female embryos (Imp2^{♀+/♂+}) They also exhibited impaired embryonic developmental configurations (FIGS. 3A and 3C). Further observation indicated a slight increase in the 4-cell-stage embryo rate (13%) in Imp2^{-/-} females at 62 h post-hCG (FIG. 3A). Only 6% of the Imp2^{♀-/♂+} embryos developed into the blastocyst stage compared to 82% of the control embryos (FIG. 3B). Most embryos died prior to compaction or had fragmented into cytoplasmic blebs (FIG. 10D). In vivo and in vitro monitoring produced consistent results regarding embryonic growth (FIG. 3B and FIG. 10C). To determine the role of an intact paternal Imp2 allele, an Imp2-knockout male germline was developed. Imp2^{-/-} males with normal fertility and spermatogenesis were used for breeding with Imp2^{-/-} females. Pregnant females were sacrificed at 3.5 days post coitus, and no significant effect was observed in blastocyst percentage after deletion of paternal Imp2 (FIG. 3B). Thus, Imp2-deletion in male mice had no effect on embryonic development, and these findings suggest the essential role for Imp2 in pre-implantation embryonic development.

We further investigated the fertility of Imp2^{-/-} and control females greater than 5 weeks old when mated with normal adult wild-type males over a period of 6 months. Imp2^{-/-} females were sub fertile and produced only a few pups during the indicated time period compared with control females (FIG. 3D). In the first one or two litters, Imp2^{-/-} females produced four or five pups, but this number gradually decreased until the mice became infertile (FIG. 3D). Thus, Imp2 is crucial for mouse female fertility.

### Example 6 Deletion of Imp2 Downregulates Target Gene Expression During Zygotic Genome Activation

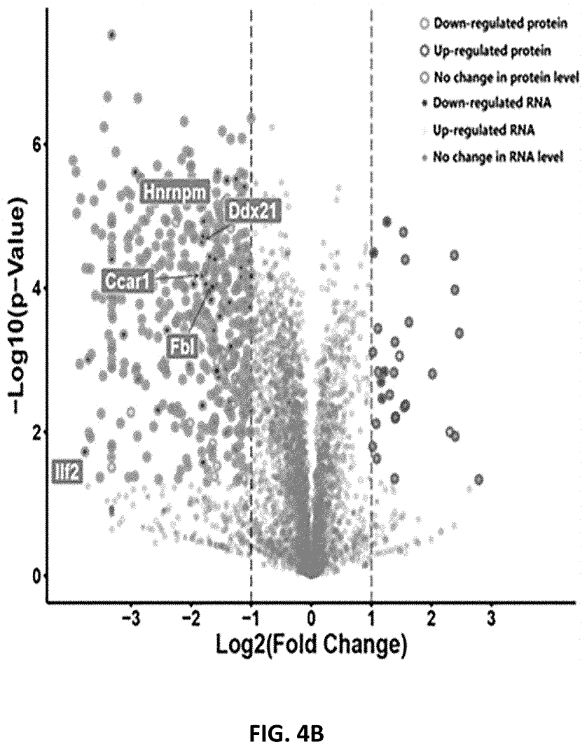

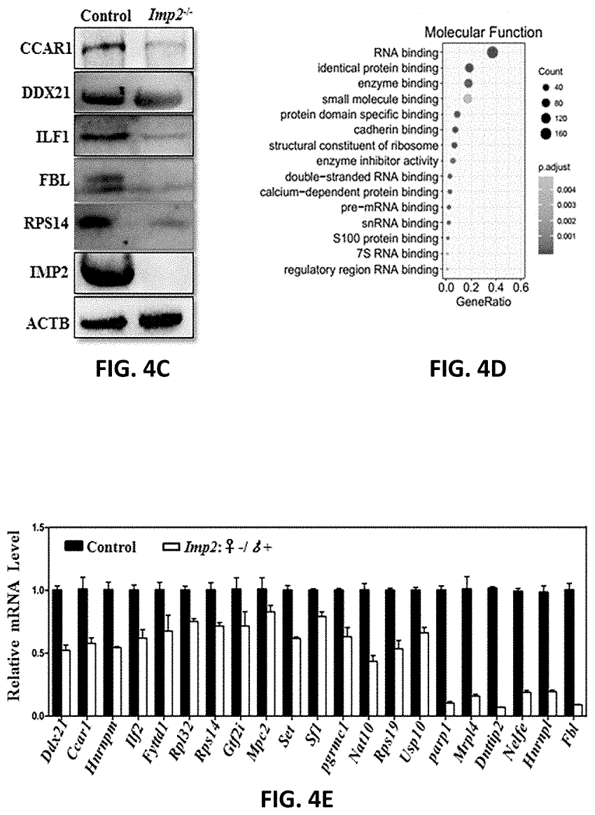

During the growth of oocytes, meiotic progression in transcriptionally silent oocytes coordinated with translation of some maternal transcripts³⁶. This synchronization is essential for the maturation of oocytes and supporting the early embryonic preimplantation development. Therefore, to identify the genes that are regulated by IMP2 in early-stage mouse embryos, we used RNA sequencing and HPLC MS/MS to study the transcriptomes and proteomes of late 2-cell-stage embryos derived from control and Imp2^{-/-} females after matting with wild-type males (FIG. 4A). RNA-sequencing identified 1,646 upregulated transcripts and 1,703 downregulated transcripts in the embryos from Imp2^{-/-} females compared to those from wild-type females, while HPLC MS/MS analysis identified 32 upregulated proteins and 285 downregulated proteins (FIG. 4A). The data from the transcriptome and proteome analyses were merged to further identify the downregulated targets. A total of 34 transcripts were screened out after merging the RNA-sequencing and HPLC MS/MS data (FIG. 4A). Further, 18 downregulated gene were found after merging the transcripts obtained from RNA-sequencing and HPLC MS/MS merged data by photoactivatable ribonucleoside-enhanced crosslinking and immunoprecipitation (FIG. 4A). We found that knockout of Imp2 inhibited the expression of target genes and resulted in greater downregulation than upregulation of proteins and RNAs (FIG. 4B). Western blot validation was consistent with the downregulation seen in the RNA-protein merged data (FIG. 4C). These enriched downregulated genes with reduced protein expression were primarily involved in RNA-binding and protein-binding activities (FIG. 4D).

A selection of transcripts was measured by qRT-PCR. The data showed consistency with the RNA-sequencing and HPLC MS/MS data (FIG. 4E), which suggests that the cause of defective embryogenesis is due to the deletion of maternal Imp2.

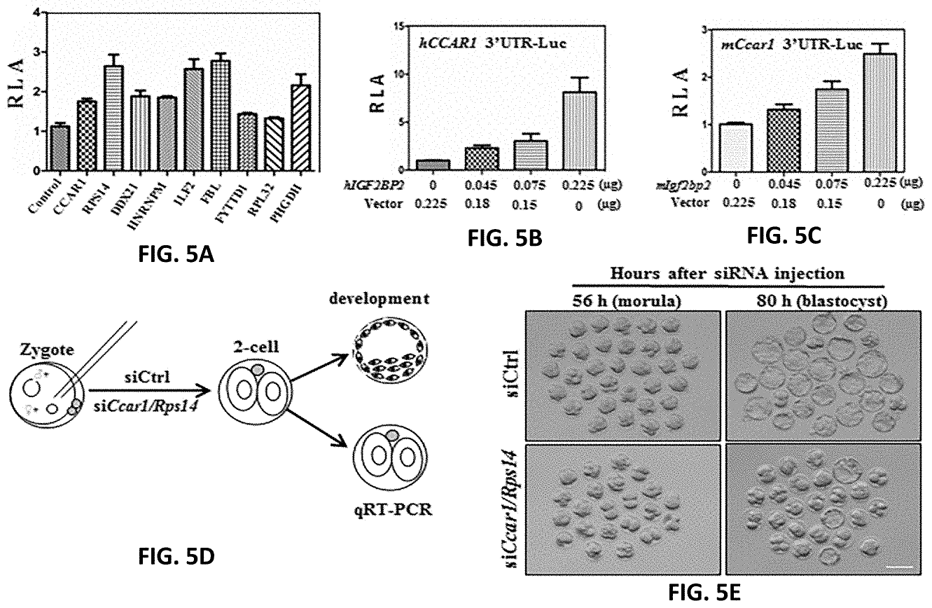

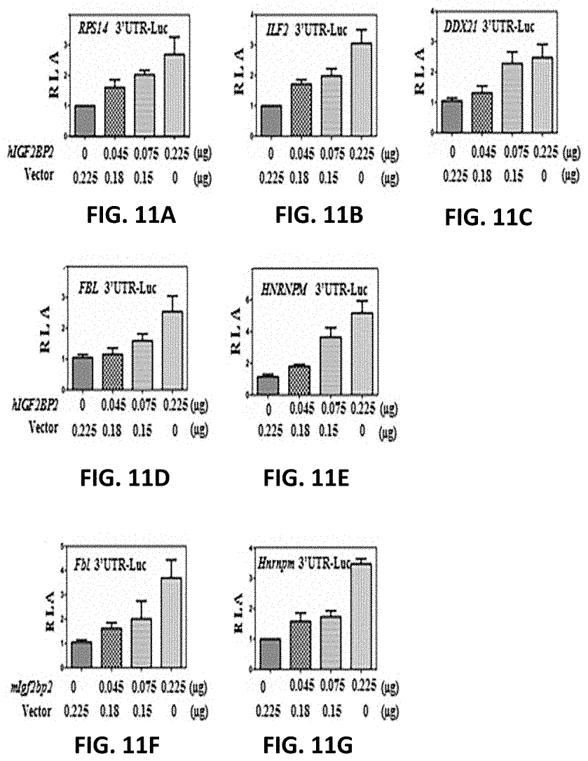

To determine the IMP2 target genes during embryonic growth, 9 downregulated genes were selected among 18 candidates from the RNA-protein merged data and qRT-PCR validation (FIG. 5A). Among them, Ccar1 and Rps14 were found to be the target genes for IMP2 (FIG. 5B, FIG. 5C and FIG. 11A). Gene ontology (GO) analysis revealed that these genes are highly enriched in RNA binding and metabolic processes and are crucial for early embryonic developmental competence. The induction of these two genes might be required for early embryonic development by increasing RNA-binding and metabolic activity. Combined deletion of Ccar1 and Rps14 decreased embryonic development compared to WT embryos (FIGS. 5D-5F), and the embryos degraded prior to compaction (FIG. 5E). Reduced mRNA expression was observed in Ccar1 and Rps14-depleted embryos as indicated by qRT-PCR analysis (FIG. 5G). Collectively, these results suggest that IMP2 is expressed in early embryos and that it activates the transcription of Ccar1 and Rps14.

To determine whether IMP2 alters translational activity, a luciferase reporter assay was performed in a dose-dependent manner on defined transcripts. The translation profile was monitored with a dual luminescence assay in relation to increasing amounts of Igf2bp2 and increased luciferase activity was observed in a dose-dependent manner (FIG. 5B, FIG. 5C and FIGS. 11A-11G). GO analysis revealed that the downregulated genes are associated with poly(A) RNA binding, RNA splicing, and RNA transport. These results suggest that the upstream activity of genes linked with Imp2 support the developmental competence of early-stage embryos by increasing the translation of genes associated with RNA-binding activity.

### Example 7 Imp2 Deletion Perturbs the Transcriptional and Translational Machinery in Cleaved Embryos

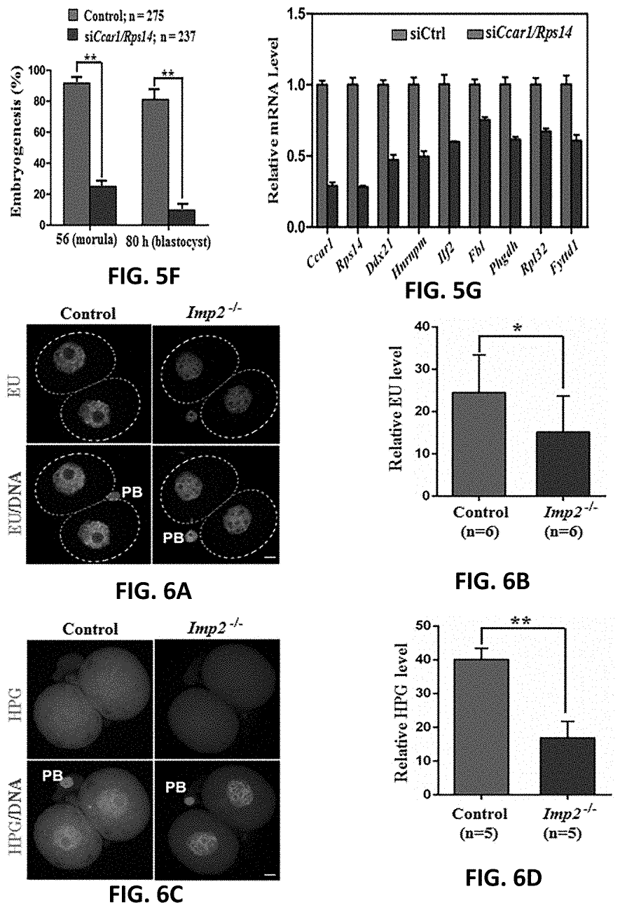

The gene expression reprogramming that is required during early embryonic preimplantation development coincides with changes in chromatin structure that are associated with RNA synthesis. To determine the role of IMP2 in transcriptional activity, we used 2-cell-stage embryo samples of both genotypes (control and Imp2^{-/-}) for an EU (ethynyl uridine) incorporation assay. EU, which is an alkyne-modified nucleotide, can be actively incorporated into nascent RNA when incubated with the oocytes and embryos. EU incorporation was greatly decreased in Imp2^{-/-} females derived 2-cell-stage embryos compared with control embryos (FIGS. 6A and 6B), and resulted in defects in transcriptional activity.

To test whether Imp2-deletion also affects total protein synthesis during ZGA, 2-cell-stage embryos were incubated in culture medium supplemented with 50 μM HPG (L-homopropargylglycine) for 2 h. HPG signal intensity is indicative of translational activity and was two times lower in IMP2-depleted, 2-cell-stage embryos compared with controls (FIGS. 6C and 6D). Taken together, our results indicate that the transcriptional and translational activity essential for gene expression during embryonic growth is IMP2 dependent.

### Example 8 Increased Early Embryonic Developmental Potential in Mice by IGF2 Supplementation

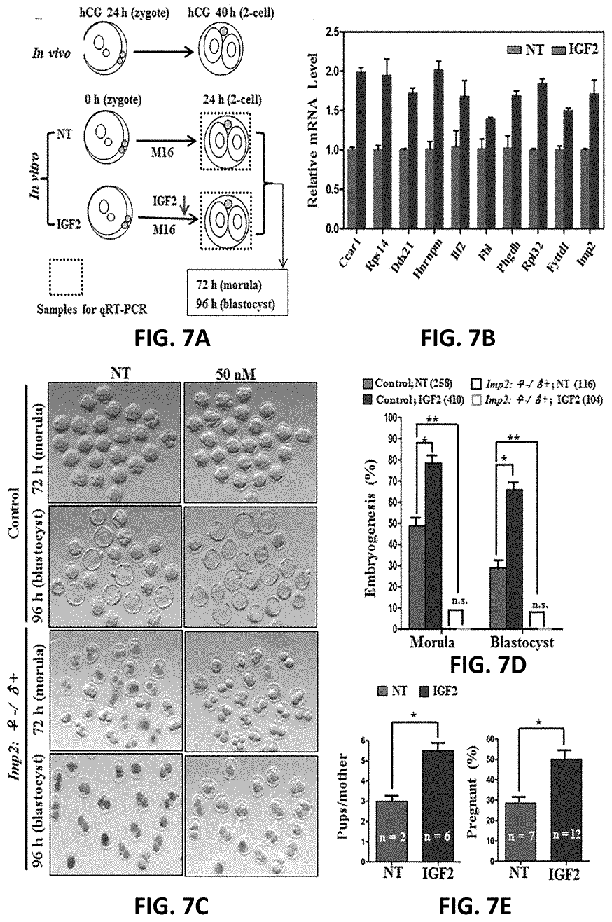

M16 is a frequently used culture medium, but it reduces the rate of embryonic development into the morula and blastocyst stages, and thus different growth factors have been added to the culture medium to improve early embryonic growth. Previously, IGF2 has been used for the maturation of porcine oocytes. To determine the functional role of IGF2 in embryonic development, zygotes were cultured in M16 medium with or without IGF2 (FIG. 7A). IGF2 treatment promoted the expression of downstream genes in cultured embryos (FIG. 7B), and adding IGF2 to the culture medium improved the development rate of controls embryos (Imp2^{♀+/♂+}), but no effect was observed in Imp2^{-/-} female derived embryos (Imp2^{♀-/♂+}) (FIGS. 7C and 7D).

Embryo transfer was performed to further investigate the developmental fate of IGF2-treated embryos in vivo. In an experiment related to embryo transfer, 12 foster mothers for IGF2-treated embryos and 7 for non-treated control embryos were used as the recipients. Foster mothers receiving IGF2-treated embryos delivered more pups per female, and their pregnancy rate was also significantly greater than females who received control embryos (FIG. 7E and FIG. 10F). These results suggest that IGF2 activates the signaling pathways that stimulate the downstream genes and thereby increase early embryonic developmental competency.

### Example 9 IGF2 is Crucial for Improving Human Embryonic Developmental Competency

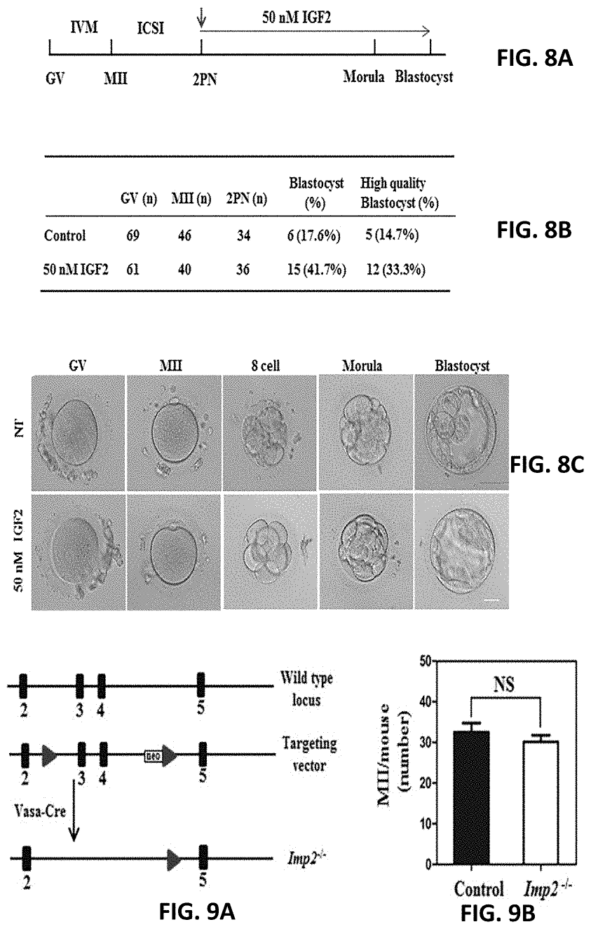

We examined the clinical application of IGF2 in human embryonic development in vitro. Zygotes were cultured in medium with or without 50 nM IGF2 after in vitro maturation and intracytoplasmic sperm injection of oocytes (FIG. 8A), and increased blastocyst formation (41.7%) was observed in IGF2-treated embryos compared to control embryos (17.6%) (FIG. 8B). Moreover, the percentage of high-quality blastocyst was higher from cultured embryos treated with IGF2 compared with controls (FIGS. 8B and 8C). Thus, adding IGF2 to the culture medium increased the rate of blastocyst formation along with improved quality, and this suggests the potential for the clinical application of IGF2 in human assisted-reproduction techniques.

### Example 10 IGF2 Improves the Developmental Competency and Meiotic Structure of Oocytes from Aged Mice

Materials and Methods

Mice

Young (4 weeks) and aged (42-45 weeks old) ICR female mice (Charles River Laboratories China Inc) were selected for this experiment. All animal experimental protocol was performed accordance to the ethical guidelines approved by the Animal Care and Research Committee of Shandong University.

Oocytes Collection and Culture

To get fully grown GV-stage oocytes, aged mice were superstimulated with 5 IU pregnant mare's serum gonadotropin (PMSG) injection. After 48 h of PMSG injection, cumulus oocytes complex were obtained by manually rupturing the ovarian follicles structure. The oocytes were collected and randomly divided into two groups. Oocytes with or without 50 nM IGF2 (100-12, Peprotech), were cultured in the small drops of M16 (M7292; Sigma-Aldrich), and maintained in 5% CO₂ at 37° C. For collection of MII-stage oocytes, mice received an injection of 5 IU PMSG followed by 5 IU human chorionic gonadotrophin (hCG) after 44 h. MII-stage oocytes were collected after 16 h of hCG and used for in vitro fertilization (IVF) experiment.

Zygotes Culture and Embryo Transfer

MII-stage oocytes were collected and IVF experiment was performed by using sperms from wild-type (WT) male. Zygotes were cultured in M16 medium with or without 50 nM IGF2, and incubated at 37° C. in 5% CO₂ for observing their embryonic developmental competence. Embryos development and morphology were examined with a stereomicroscope (Nikon SMZ1500). In an experiment related to embryo transfer, blastocysts obtained with or without IGF2-treatment were transferred. WT female mice were used as the recipients (15 embryos were transferred to the uterus of each mouse), and pregnancy rates to term were recorded.

Estimation of Serum IGF2 Concentration

The concentration of IGF2 was measured in mouse serum samples by following the manufacturer's instructions using ELISA kit (RnD system, MG200). Briefly, blood from young and aged mice were collected and put at room temperature for 1 h. Samples were centrifuged at 3000×g for 10 min at 4° C. Serum was collected and stored at −80° C. for subsequent assay. The IGF2 concentration was determined in triplicate. The standard curves were generated, and the IGF2 content was calculated using the formula derived from the standard curve.

RNA Extraction and qRT-PCR Validation

Total RNA was extracted using RNeasy mini kit (Qiagen) following the manufacturer's instructions. Genomic DNA (gDNA) was eliminated by digesting with RNase-free genomic DNA eraser buffer (Qiagen), and cDNA was obtained by reverse transcription of RNA using PrimeScript™ reverse transcriptase (Takara). Power SYBR Green Master Mix (Takara) was used on a Roche 480 PCR system for qRT-PCR analysis. The qRT-PCR reactions were performed in triplicate for gene specific primers. The mRNA level was calculated by normalizing to the endogenous mRNA level of actin (internal control) using Microsoft Excel. Primer sequences are shown in Table 4).

TABLE 4
Primer sequences for qRT-PCR.

| | Forward | Reverse |
|---|---|---|
| IGF22 | TTCTACTTCAGCAGGCCTTCAA | ATATTGGAAGAACTTGCCCACG |
| | (SEQ ID NO: 51) | (SEQ ID NO: 52) |
| SIRT1 | CTGTTGACCGATGGACTCCT | GCCACAGCGTCATATCATCC |
| | (SEQ ID NO: 53) | (SEQ ID NO: 54) |
| BMP15 | TCCTTGCTGACGACCCTACAT | TACCTCAGGGGATAGCCTTGG |
| | (SEQ ID NO: 55) | (SEQ ID NO: 56) |
| GDF9 | TCTTAGTAGCCTTAGCTCTCAGG | TGTCAGTCCCATCTACAGGCA |
| | (SEQ ID NO: 57) | (SEQ ID NO: 58) |
| SOD1 | GCTGTACCAGTGCAGGTCCTCA | CATTTCCACCTTTGCCCAAGTC |
| | (SEQ ID NO: 59) | (SEQ ID NO: 60) |

Immunofluorescence

To detect relevant protein, the oocytes were fixed in 4% paraformaldehyde for 30 min, permeabilized with 0.3% Triton X-100 for 20 min. After washing three times, the oocytes were blocked in blocking buffer in PBS with 1% BSA. Oocytes were incubated with a fluorescein isothiocyanate (FITC)-conjugated anti-mouse Alpha tubulin (1:200 dilution, Sigma) antibody, anti-γ-H2AX (1:300 dilution, Abcam), anti-apoptotic (1:1000 dilution, Abcam), and anti-LC3 (1:300, Abcam) for 1 h at room temperature. After washing three times, oocytes were incubated with respective secondary antibodies. DNA was counterstained with DAPI (Sigma) for 10 min at room temperature. Oocytes were washed and mounted on the glass slides and observed under confocal laser microscope (Zeiss LSM 780, Carl Zeiss AG, Germany).

Determination of ATP Levels

The measurement of total ATP content of MII-stage oocytes obtained with and without IGF2-treatment was performed by using ATP testing assay kit (Beyotime). Briefly, 50 oocytes were added to lysis buffer and centrifuged at 12000×g for 10 min. Supernatant was mixed with testing buffer, and ATP concentrations were measured on a luminescence detector (EnSpire Multimode Plate Reader). A 6-point standard curve was generated ranging from 0.01 mM to 1 m and total ATP contents were calculated.

ROS Evaluation

ROS was measured in MII-stage oocytes by using ROS assay kit (Beyotime) by following manufacturer's instructions. Briefly, control and IGF2-treated oocytes were incubated with 10 μM, 2′,7′ dichlorofluorescein diacetate (DCFH-DA) in M16 medium at 37° C. in 5% CO₂ for 30 minutes. After three washes, oocytes were mounted on glass slides, and examined under confocal laser microscope (Zeiss LSM 780, Carl Zeiss AG, Germany).

Detection of Mitochondrial Distribution and JC-1 Assay

To detect mitochondrial distribution, MII-stage oocytes were incubated with 400 nmol/L Mito tracker Green FM (Invitrogen) diluted in PBS for 30 minutes at 37° C. and fixed in 2% paraformaldehyde for 20 minutes. To evaluate the mitochondrial membrane potential, the oocytes were incubated in M16 culture medium containing 10 μM JC-1 (Beyotime Institute of Biotechnology) at 37° C. for 30 min. After washing three times in PBS, the oocytes were mounted on glass slides and observed immediately (Zeiss LSM 780, Carl Zeiss AG, Germany). The red and green fluorescents intensities were determined and mitochondrial membrane potential was calculated as the ratio of red and green fluorescent pixels.

Detection of Protein Synthesis

The protein synthesis assay was performed using the Click-iT protein synthesis assay kit (C10428, Life Technologies) following the manufacturer's instructions. Briefly, the MII-stage oocytes were incubated in culture medium supplemented with 50 μM HPG at 37° C. with 5% CO2 for 1 h. Oocytes were fixed with 3.7% formaldehyde followed by permeabilization with 0.5% Triton X-100 for 20 min at room temperature. The HPG signal is indicative of the overall level of translation in oocytes.

Electron Microscope

Briefly, MII-stage oocytes treated with or without IGF2 were collected, visualized and captured with a transmission electron microscope (TEM, JEOL). The numbers of normal and vacuolated mitochondria were quantified in defined region of interests (ROIs) in the oocyte cytoplasm using IMAGE J (National Institutes of Health, USA).

Statistical Analysis

Data are presented as mean±SEM of three independent experiments/samples unless otherwise specified. Group comparisons were made by two-tailed unpaired Student's I-tests. *p<0.05; **P<0.01, and ***P<0.001. All analyses were performed using the GraphPad Prism (GraphPad Software, San Diego, Calif., USA).

### Experiments and Results

1. Aged Mice have Reduced Serum IGF2 Protein Levels and their Oocytes have Reduced Igf2 Expression

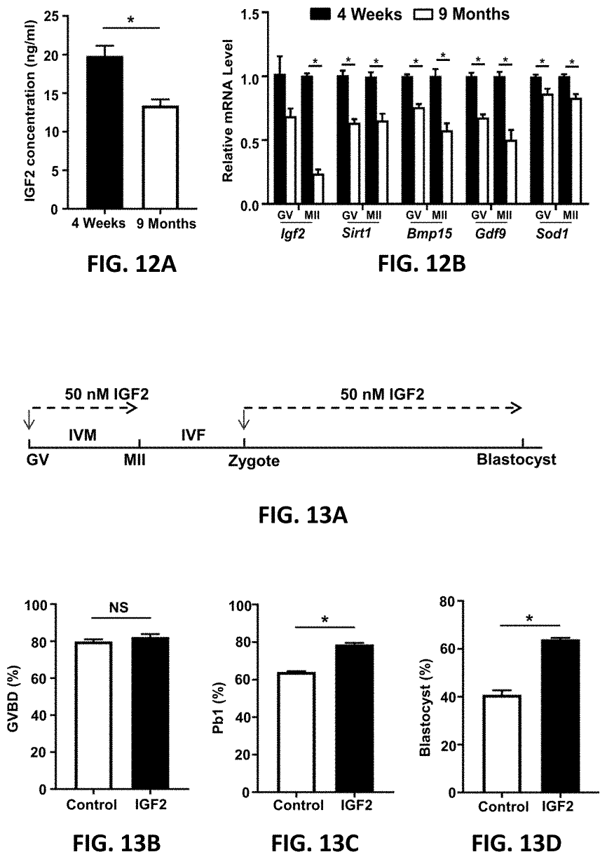

In light of previous reports of fertility-promoting roles for IGF2, we investigated the potential involvement of this growth factor in oocyte development in aged mice of 9 months. We first evaluated the IGF2 level in blood sera samples from young (4 weeks) and aged (9 months) mice using ELISA, which revealed that the aged mice had significantly reduced IGF2 concentrations (FIG. 12A). Further associating an age-related decline in IGF2 levels with age-related declines in fertility, a qPCR analysis of GV-stage and MII-stage oocytes retrieved from young and aged mice also revealed reductions in the mRNA levels of Igf2 (FIG. 12B). Further, we detected significant reductions in the levels of known antioxidant and oocyte-specific genes, including Sirt1, Bmp15, Gdf9, and Sod1 (FIG. 12B). Collectively, these findings suggest that reduced IGF2 levels may be involved in the impaired oocyte development known to occur in aged mice.

2. Treatment of Oocytes from Aged Mice with IGF2 Improves Meiotic Maturation and Early Embryonic Development

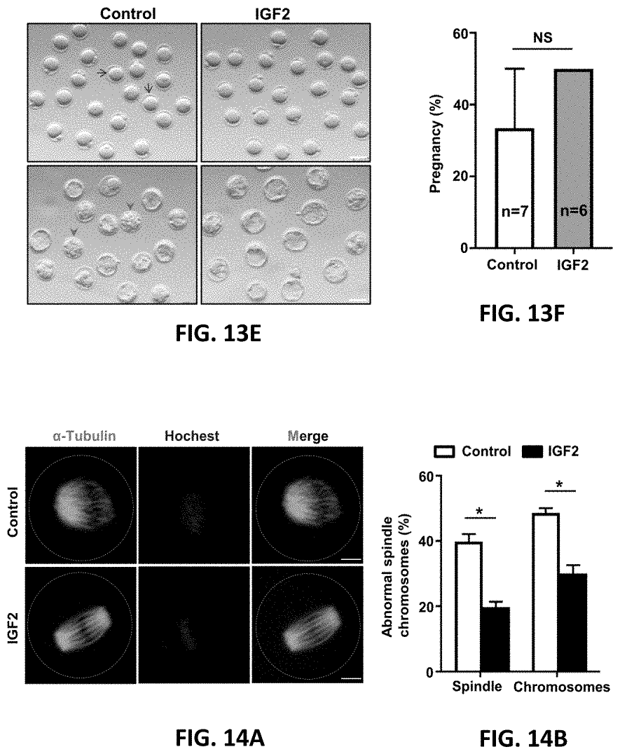

To investigate whether IGF2 supplementation in culture media functionally impacts oocytes development in aged mice, GV-stage oocytes were collected from aged mice and cultured in medium with or without 50 nM IGF2 (FIG. 13A). We observed that the presence of IGF2 had no effect on meiotic resumption; as no difference in the percentage of germinal vesicle breakdown (GVBD) was noticed after 3 h of in vitro culture (FIG. 13B). However, IGF2 increased the polar body (Pb1) extrusion rate significantly (p<0.05) (FIGS. 13C and 13E). We observed a significant increase in oocyte maturation in the presence of IGF2: whereas a majority of the control oocytes arrested at the GVBD-stage, more than 79% percent of the IGF2-exposed oocytes proceeded into the MII-stage (FIGS. 13C and 13E).

We additionally explored potential functional impacts of IGF2 on embryonic development by culturing zygotes from aged mice in M16 medium with or without 50 nM IGF2. The presence of IGF2 in the culture medium increases the proportion of zygotes that developed into blastocysts: from 41% in the untreated control group to 64% in the IGF2 group (p<0.05) (FIGS. 13D and 13E). Note that most of the embryos in control group arrested at the compact morula-stage (FIG. 13E). We also examined developmental-fate-related effects of IGF2-treatment in vivo with an embryo transfer experiment which showed that pregnancy rates did not differ between control and IGF2-treated embryos (FIG. 13F). These results suggest that IGF2 does not apparently enhance embryonic development in vivo. Thus, our data suggest that IGF2 may have the potential to improve the meiotic maturation and early embryonic developmental competency of oocytes from aged mice.

3. IGF2 Promotes the Spindle Assembly and Chromosome Alignment while Also Reducing ROS Levels in Aged Mouse Oocytes

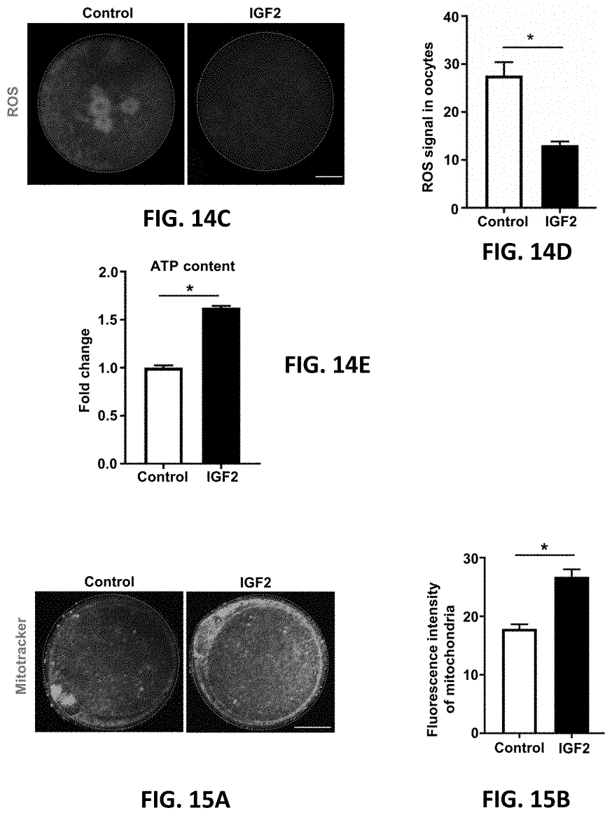

We investigated whether administration of IGF2 during in vitro culture could improve the quality of oocytes from aged mice. Specifically, we retrieved immature GV-stage oocytes from aged mice and cultured them in M16 medium with or without 50 nM IGF2 until MII-stage. Immunofluorescence analysis of MII-stage oocytes revealed that the IGF2 treatment resulted in a significant reduction in both spindle and chromosomal alignment abnormalities (FIGS. 14A and 14B). We found that the majority of the IGF2-treated oocytes displayed typical barrel-shaped spindles with well-aligned chromosomes (FIG. 14A). In addition, we found that the ROS level was significantly reduced in the IGF2-treated oocytes compared to controls (FIGS. 14C and 14D) and also detected significantly increased ATP content in the IGF2-treated oocytes (FIG. 14E). Collectively, these in vitro results show that IGF2 can improve the quality of oocytes from aged mice, specifically by promoting spindle assembly and chromosomes alignment and by reducing ROS levels.

4. IGF2 Improves Mitochondrial Function in Oocytes from Aged Mice

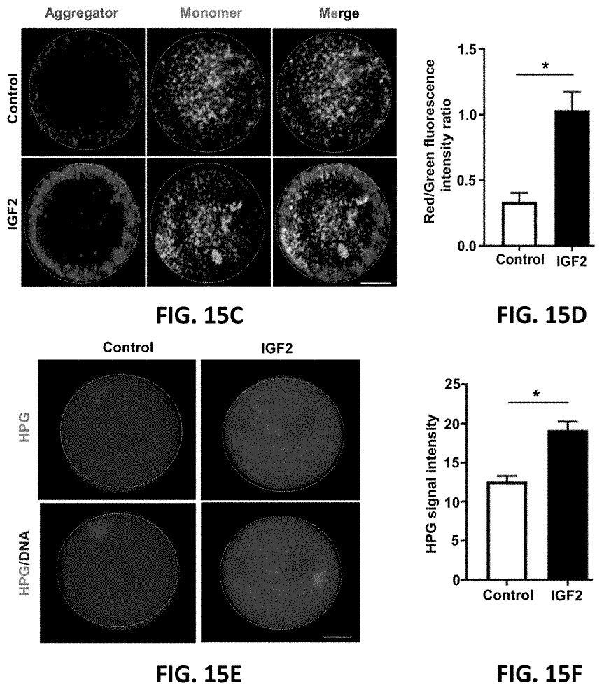

We examined the impacts of IGF2 on mitochondrial function in oocytes from aged mice with experiments wherein in vitro-matured MII-stage oocytes were cultured with or without IGF2. Immunofluorescence analysis revealed that IGF2 treatment resulted in significantly increased immunofluorescence staining intensity for mitochondria: higher fluorescence intensity of Mitotacker Green FM was observed in IGF2-treated oocytes compared to un-treated control oocytes (FIGS. 15A and 15B). Moreover, JC-1 staining assays revealed that treatment of aged mouse oocytes with IGF2 increased the MMPs index (FIGS. 15C and 15D), clearly indicating a role for IGF2 in somehow promoting mitochondrial function in aged oocytes.

To test whether IGF2 administration could improve global protein synthesis in oocytes from aged mice, control and IGF2-treated MII-stage oocytes were incubated in a medium containing L-homopropargylglycine (HPG, a methionine analogue that is incorporated into nascent proteins during active protein synthesis) for 1 h at 37° C. HPG signals are indicative of overall translational activity, and our data revealed that administration of IGF2 in culture medium could improve the translation activity in oocytes from aged mice: increased HPG signal intensity was detected in IGF2-treated oocytes relative to control oocytes (FIGS. 15E and 15F). Taken together, these results suggest that administration of IGF2 can activate mitochondrial function in a way that consequently improves the quality of oocytes from aged mice.

5. IGF2 Improves the Ultrastructure of Mitochondria of Oocytes from Aged Mice

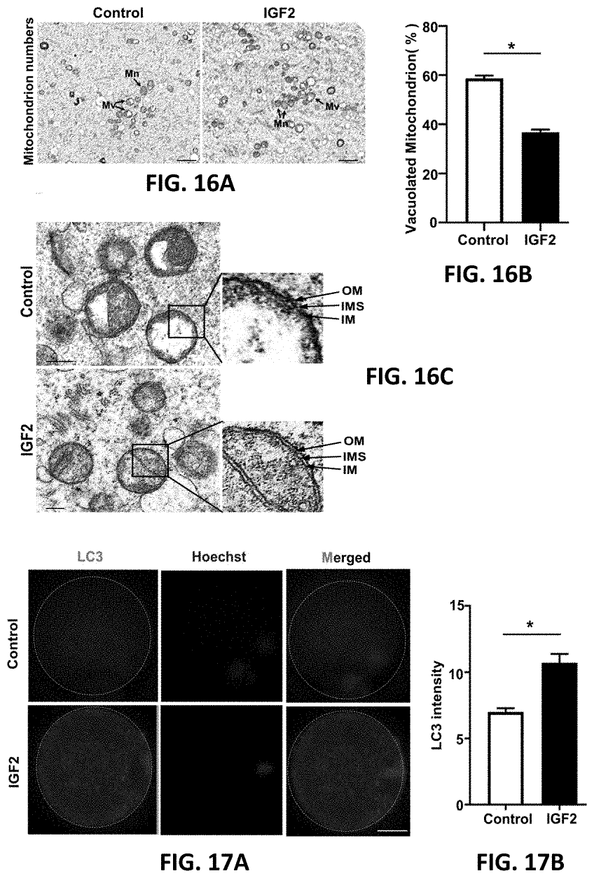

Given our finding that IGF2 administration mediates the functional activity of mitochondria, we next assessed whether IGF2 supplementation exerts any functional impact(s) on the ultrastructure of mitochondria in oocytes from aged mice. Transmission electron microscopy of MII-stage oocytes from aged mice revealed a normal morphology for mitochondria shape, with defined cristae in IGF2-treated oocytes; in contrast many mitochondria in un-treated control oocytes had vacuolated cristae (FIGS. 16A and 16B). Most IGF2-treated oocytes had mitochondria with clearly visible intact inner membranes, outer membranes, and well-defined intermembrane spaces, whereas un-treated control oocytes contained many ruptured and discontinuous inner and outer membranes with disrupted intermembrane structures (FIG. 16C). Thus, IGF2 treatment can improve the ultrastructure of mitochondria in oocytes from aged mice.

6. IGF2 Promotes the Autophagy and Also Reduces the Apoptotic Index of Oocytes from Aged Mice

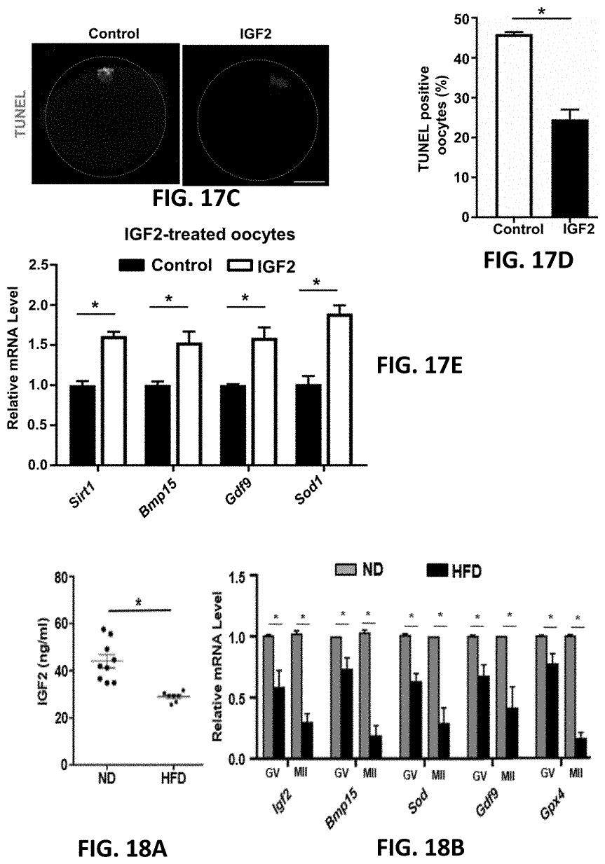

Autophagy is an essential cellular process that degrades degenerated proteins and cellular organelles to recycle their components in the cytoplasm. We examined whether supplementation with IGF2 may promote autophagy in aged mouse oocytes in experiments using the total LC3 level as an indicator for autophagy activity. The autophagy index of oocytes from aged mice was significantly increased by supplementation with IGF2 in the culture medium compared to controls (FIGS. 17A and 17B).

We checked whether IGF2 supplementation of culture medium has any impact(s) on the extent of oocyte apoptosis in aged mice, and found that IGF2-treatment significantly reduced apoptosis compared to controls after 16 h of culturing (FIGS. 17 C and 17D).

We found that administration of IGF2 to the culture medium significantly induced the expression of genes including Sirt1, Bmp15, Gdf9, and Sod1 in oocytes from aged mice compared to controls (FIG. 17E). Overall, these results suggest that IGF2 can maintain the autophagy level and can reduce the apoptotic index of oocytes from aged mice.

### Example 11 IGF2 Improves the Developmental Competency and Meiotic Structure of Oocytes from Obese Mice

1. Establishment of Obese Mice Model Induced by High-Fat Diet

To develop mice model of obesity, ICR female mice were fed with high-fat diet (HFD) for the period of 12 weeks that started from the age of 4 weeks. To establish control mice, normal diet (ND) was provided to the mice for the same time period with same age. These mice are named as “HFD mice and “ND mice” respectively. The data has shown that female mice received the HFD became obese and their average body weight is significantly higher relative to ND mice. The HFD mice indicated glucose intolerant and insulin resistant at different time point that was evaluated by glucose tolerance test (GTT) and insulin tolerance test (ITT) respectively. Therefore, these mice were used for the following experiments.

2. Obese Mice have Reduced Serum IGF2 Protein Levels and their Oocytes have Reduced Igf2 Expression:

On the basis of previous reports of fertility-enhancing roles for IGF2, we investigated the potential involvement of this growth factor in oocyte development from obese mice. We first evaluated the IGF2 level in blood sera samples from ND and HFD mice using ELISA, which revealed that the HFD mice had significantly reduced IGF2 concentrations (FIG. 18A). Further associating an obesity-related decline in IGF2 levels with obesity-related declines in fertility, a qPCR analysis of GV-stage and MII-stage oocytes retrieved from ND and HFD mice also revealed reductions in the mRNA levels of Igf2 (FIG. 18B). In addition, we detected significant reductions in the levels of known antioxidant and oocyte-specific growth indicator genes, including Bmp15, Sod1, Gdf9, and Gpx4 (FIG. 1B). Collectively, these findings suggest that reduced IGF2 levels may be associated with impaired oocyte development known to occur in obese mice.

3. Treatment of Oocytes from Obese Mice with IGF2 Improves Early Embryonic Development

To investigate whether IGF2 supplementation in culture media functionally impacts oocytes development in obese mice, GV-stage oocytes were collected from HFD mice and cultured in medium with or without 50 nM IGF2. We observed that the presence of IGF2 had no effect on meiotic maturation; as no significant difference (p>0.05) in the percentage of germinal vesicle breakdown (GVBD) and the polar body (Pb1) extrusion rate was noticed after 16 h of in vitro culture.

We additionally explored potential functional impacts of IGF2 on embryonic development by culturing zygotes from HFD mice in M16 medium supplemented with or without 50 nM IGF2. The presence of IGF2 in the culture medium increases the proportion of zygotes that developed into blastocysts. We also examined the quality of embryos treated with IGF2 by counting the inner cell mass (ICM) and trophectoderm (TE) of blast stage embryos. The data indicated increased ICM and TE in embryos treated with IGF2 relative to non-treated embryos.

4. IGF2 ameliorates spindle and chromosome defects while also reducing ROS levels in oocytes from obese mice.

We investigated whether administration of IGF2 in the culture medium could improve HFD oocytes quality by reducing these meiotic defects. Specifically, we retrieved GV-stage oocytes from ND and HFD mice, and cultured HFD mice derived oocytes in M16 medium with or without 50 nM IGF2 until MII-stage. These oocytes were immunolabeled with antitubulin antibody and counterstained with Hoechst to observe the spindle assembly and chromosomes alignment respectively. Confocal microscopy coupled with quantitative analysis of MII-stage oocytes revealed that the IGF2 treatment resulted in a significant reduction in both spindle and chromosomal alignment abnormalities in oocytes from obese mice. HFD oocytes treated with IGF2 displayed typical barrel-shaped spindles with well-aligned chromosomes compared to the HFD oocytes without IGF2-treatment.

In addition, IGF2 supplementation to the culture medium significantly increased (p<0.05) the ATP contents of HFD oocytes. Furthermore, we found that the ROS level was significantly reduced in the IGF2-treated HFD oocytes compared to HFD oocytes without IGF2 treatment. Collectively, these in vitro results show that IGF2 can improve the quality of oocytes from obese mice, specifically by promoting spindle assembly and chromosomes alignment and by reducing ROS levels.

1. An embryo culture medium for culturing an early stage embryo of a mammal, comprising about 10-200 nM IGF2.

2. The embryo culture medium according to claim 1, comprising about 45-55 nM IGF2.

3. The embryo culture medium according to claim 2, comprising about 50 nM IGF2.

4. The embryo culture medium according to claim 1, wherein the early stage embryo is a 2-cell stage embryo, a 4-cell stage embryo, an 8-cell stage embryo, a morula, or a blastocyst.

5. The embryo culture medium according to claim 1, further comprising a background medium.

6. The embryo culture medium according to claim 5, wherein the background medium is M2 or M16 medium.

7. The embryo culture medium according to claim 1, wherein the mammal is a mammal of rodentia, lagomorpha, carnivora, artiodactyla, perissodactyla, or of primate, and simian.

8. The embryo culture medium according to claim 7, wherein the mammal is a human being, a monkey, a rat or a mouse.

9. The embryo culture medium according to claim 1, wherein the early stage embryo is from an aged mammal.

10. The embryo culture medium according to claim 1, wherein the early stage embryo is from a mammal with obesity.

11. A method for culturing a mammalian embryo in vitro, comprising: culturing an early stage embryo of a mammal in an embryo culture medium comprising about 10-200 nM IGF2.

12. The method according to claim 11, wherein the embryo culture medium comprises about 45-55 nM IGF2.

13. The method according to claim 11, wherein the embryo culture medium comprises about 50 nM IGF2.

14. The method according to claim 11, wherein the early stage embryo is in a 2-cell embryo stage, a 4-cell embryo stage, an 8-cell embryo stage, a morula stage or a blastocyst stage of the embryo.

15. The method according to claim 14, wherein the early stage embryo is in a 2-cell embryo stage.

16. The method according to claim 11, wherein the embryo culture medium further comprises a background medium.

17. The method according to claim 16, wherein the background medium is M2 or M16 medium.

18. The method according to claim 11, wherein the mammal is a human being, a monkey, a rat or a mouse.

19. The method according to claim 11, wherein the early stage embryo is from an aged mammal.

20. The method according to claim 11, wherein the early stage embryo is from a mammal with obesity.
